(19) United States
(12) Patent Application Publication
(10) Pub. No.: US 2018/0153819 A1
(43) Pub. Date: Jun. 7, 2018

(54) Nanocapsule utilizing mutant chaperonin complex for system of local drug delivery into cell

(71) Applicant: School Judicial Person Ikutoku Gakuen, Kanagawa (JP)

(72) Inventors: Ayumi Koike, Kanagawa (JP); Hiromi Yoda, Kanagawa (JP); Takeji Takamura, Kanagawa (JP)

(21) Appl. No.: 15/573,795

(22) PCT Filed: May 11, 2016
(86) PCT No.: PCT/JP2016/063939

§ 371 Date: Nov. 13, 2017

(30) Foreign Application Priority Data
May 16, 2015 (JP) 2015-100586

(51) Int. Cl. A61K 9/51; A61K 47/42

(52) U.S. Cl. CPC A61K 9/5169; A61K 47/42

(57) ABSTRACT

An object is to provide a technology that relates to a protein nanocapsule capable of holding a substance to be encapsulated such as a drug and in which the protein nanocapsule can be introduced into a cell using a simple method and the encapsulated substance can reach a target in a cell.

The present invention relates to a nanocapsule for a drug delivery system including, as a carrier material for encapsulation of a pharmacological component for a nanocapsule for a system of local drug delivery into a cell, a mutant chaperonin complex including an ATP hydrolysis activity-lowered GroEL subunit mutant as a GroEL subunit included in a ring structure and a subunit having GroES activity as a subunit included in an apex portion.

### CROSS REFERENCE TO RELATED APPLICATIONS

This application is a Section 371 National Stage Application of International Application No. PCT/JP2016/063939, filed May 11, 2016, and published as WO/2016/185955 A1 on Nov. 24, 2016, which claims priority to and benefits of Japanese Patent Application Serial No. 2015-100586, filed with the Japan Patent Office on May 16, 2015, the entire contents of which are incorporated herein by reference.

### TECHNICAL FIELD

The present invention relates to a nanocapsule for a drug delivery system that utilizes a mutant chaperonin complex and can carry out local delivery into a cell.

### BACKGROUND ART

Current examples of drug administration means that are commonly used include transdermal administration, intravenous administration, and oral administration. However, in these cases, a drug is systemically administered while circulating systemically, and therefore, its local efficacy cannot be expected. Accordingly, when high-concentration administration or the like is required in order to obtain sufficient efficacy, dangers brought on by issues including side-effects and the like on non-target organs and tissues often arise.

Therefore, in technical fields of pharmaceuticals, medicine, and the like, drug delivery systems (DDS) are being vigorously studied as next-generation drug administration methods, and technology relating to locality, cell-membrane penetration property, and the like that enable a drug to reach only its target cell have been extensively developed.

Regarding a carrier used in a drug delivery system, there have been many studies in which an endocytotic function of a cell is utilized so that a drug-containing liposome is taken up by the cell (Non-Patent Document 1). In order to use a liposome as a DDS carrier, it is possible to provide a ligand that binds to a membrane receptor, an antibody that recognizes a protein exposed on a cell surface, or the like, that is, a means in which a protein is used in a portion to be recognized by a cell is conceivable.

However, although it is desirable that a drug delivery carrier has a size such that it can pass through a capillary vessel and is uniform in size, there is a problem in that it is difficult to adjust the particle diameter of a liposome to be uniform. Moreover, when an antibody is attached to a liposome, the particle diameter of the liposome increases, and there is a possibility that it becomes difficult for the liposome to pass through a capillary vessel. Furthermore, efficient binding of a liposome to an antibody is not easy to achieve.

An improvement in the structure of a liposome itself and an opening/closing controlling means using a specific apparatus such as an ultrasonic generator are required for the local delivery of a drug or the like contained in a liposome (Non-Patent Document 2, for example), and it is difficult to realize a simple means for opening/closing control.

Also, there is a problem in that an operation of modifying the surface of a liposome using an antibody or the like is troublesome.

Therefore, the technical development of a DDS carrier that will replace liposomes has been in demand to realize a drug delivery system characterized by localization and cell penetration properties.

A chaperonin complex (GroEL/GroES complex) is a nanocapsule-shaped protein having a three-dimensional structure that has a cavity with a diameter of about 4 to 8 nm (about 5 nm in a case of wild-type E. coli) and that is stable and uniform in an aqueous solution. Moreover, the chaperonin complex is a protein, and therefore, the addition or binding of peptides or the like thereto is easily achieved.

Accordingly, the chaperonin complex is a molecule that is attracting attention as a candidate for a DDS carrier that will replace liposomes.

Here, a chaperonin is one of the so-called molecular chaperones that assist correct folding of substrate proteins. Chaperonin family members have common characteristics in that they have a molecular weight of 50 to 60 kDa, have a ring complex structure, and assist folding of substrate proteins in an ATP dependent manner. Of the chaperonins, GroEL is a chaperonin of E. coli, and it has been revealed that GroEL assists the folding of proteins in an ATP and GroES dependent manner.

The chaperonin GroEL has a tetradecameric structure including fourteen GroEL subunits in total in which two rings that are each constituted by a GroEL-subunit heptamer are arranged back to back. A single GroEL subunit consists of an equatorial domain including an ATP binding site, an apical domain including binding sites for a substrate protein and GroES, and an intermediate domain that connects the equatorial domain and the apical domain.

In folding of a substrate protein, first, the substrate protein binds to the “entrance” of the ring constituted by the chaperonin GroEL subunits, followed by binding of seven ATPs to the respective chaperonin GroEL subunits included in the ring. As a result, the structure of the chaperonin GroEL is changed, thus making it possible for GroES, which is a cofactor, to bind to the GroEL. Subsequently, the GroES binds to the GroEL, and the substrate protein thus falls into the cavity of the ring, resulting in the formation of a chaperonin complex. In the chaperonin complex, folding of the fallen substrate protein progresses in the cavity of the ring. Next, when the ATPs in the ring are hydrolyzed, the GroES dissociates, and the folded substrate protein in the ring dissociates at the same time.

ATPs hydrolyze in about eight seconds in a normal wild-type GroEL, and this is disadvantageous in that an encapsulated substance is released in a short time. Therefore, the normal wild-type GroEL cannot be used as a drug delivery carrier as it is.

To address this, a technique has been reported in which attention is focused on the GroEL, which is a constitutional unit of the chaperonin complex, and a complex obtained by assembling GroEL-subunit heptamers in a tubular shape is formed and used to contain a substance to be encapsulated such as a drug (Non-Patent Document 3). Here, Non-Patent Document 3 reports that, similarly to a normal protein, a complex structure including GroELs, which are constitutional units of chaperonin, has difficulty in penetrating a cell membrane as it is, and a technique is reported in which a boronic acid derivative is used to modify the surface of the protein, thus enabling the complex structure including GroELs to penetrate a cell membrane.

However, the fundamental principle of the technique according to Non-Patent Document 3 is that the rings included in the GroEL tube theoretically separate from one another as a result of reacting to a high ATP concentration in a cell, and an encapsulated substance is thus released into the cell. Therefore, a drug is released from the tube “immediately after” the cell penetration, so that, with this technique, it is theoretically impossible to carry out local drug delivery to a specific intracellular organelle such as a nucleus. In this regard, it is considered that this technique is unsuitable for application to a DDS technology that enables a nucleic acid medicine to reach a target in a cell. Moreover, this technique has a problem in that an excess processing step, that is, surface modification processing using a boronic acid derivative for cell membrane penetration, is required.

Accordingly, the field of study of utilizing chaperonin as a DDS carrier is still under development, and putting the delivery of contents to an intracellular organelle (particularly to a nucleus) into practical use has not been investigated sufficiently.

As described above, a technology is expected to be developed that relates to a protein nanocapsule capable of holding a substance to be encapsulated such as a drug and in which the protein nanocapsule can be introduced into a cell using a simple method and the contained substance can reach a target in a cell, but a technology that can be put into practical use has not been developed.

### CITATION LIST

### Non-Patent Documents

Non-Patent Document 1: Patel L. N. et al., Cell Penetrating Peptides: Intracellular Pathways and Pharmaceutical Perspectives, Pharmaceutical Research (2007), 24(11), 1977-1992

Non-Patent Document 2: Yakugaku Zasshi, 130(11), p 1489-1496, 2010

Non-Patent Document 3: Biswas S. et al., Biomolecular robotics for chemomechanically driven guest delivery fueled by intracellular ATP, Nat. Chem. (2013), 5(7), 613-620

Non-Patent Document 4: Essential Cell Biology (Third Edition (Japanese Edition)), p 389

Non-Patent Document 5: Tsukazaki et al., Structure and function of a protein export-enhancing membrane component SecDF, Nature, 474 (7350), 235-238, (Nature. Author manuscript; available in PMC 2013 Jul. 1)

### SUMMARY OF INVENTION

### Technical Problem

The present invention was achieved in light of the aforementioned circumstances of the conventional techniques, and it is an object thereof to provide a technology that relates to a protein nanocapsule capable of holding a substance to be encapsulated such as a drug and in which the protein nanocapsule can be introduced into a cell using a simple method and the contained substance can reach a target in a cell.

### Solution to Problem

As a result of intensive study conducted by the inventors of the present invention in order to solve the aforementioned problems, the following findings were obtained, and the present invention was achieved.

(1) The inventors of the present invention found that, when a mutant chaperonin complex including “ATP hydrolysis activity-lowered GroEL subunit mutants” as GroEL subunits was used, the chaperonin complex itself, which serves as a nanocapsule, was taken up by a cell while holding a contained substance.

What is noteworthy here is the fact that, regardless of the report that a complex structure including GroEL subunits cannot penetrate a cell membrane as it is as described in Biswas et al. 2013 (Non-Patent Document 3), when the ATP hydrolysis activity-lowered GroEL subunit mutants were used to form a chaperonin complex including these mutant subunits and subunits having GroES activity, the cell-membrane penetration properties of a chaperonin complex were confirmed even in the chaperonin complex to which a selective cell-membrane penetrating peptide had not been added. Specifically, the inventors of the present invention found that, when the mutant chaperonin complex including the GroEL subunit mutants contained a substance to be encapsulated, the mutant chaperonin complex exhibited cytoplasm penetration properties without being subjected to processing such as special surface processing or molecular modification, while holding the above-mentioned contained substance. This is a finding that is contrary to common general technical knowledge assumed from the description in Biswas et al. 2013 (Non-Patent Document 3).

Furthermore, what is notable is the fact that this finding cannot be obtained merely by using a wild-type GroEL/GroES complex since ATPs hydrolyze in a short period of time of about eight seconds, and thus GroES and the encapsulated substance dissociate in such a short period of time in a normal wild-type GroEL. This finding was obtained for the first time by the inventors of the present invention hitting upon an idea of using the ATP hydrolysis activity-lowered GroEL mutant despite the above-mentioned negative teachings and experimentally showing that the idea can be realized.

Moreover, the fact that a macromolecule such as a normal protein (e.g., GFP) cannot penetrate a cell membrane as it is a common knowledge in the art (see Non-Patent Document 4, for example). Furthermore, it is thought that a special membrane protein structure and a specific signal are required in order for a protein to penetrate a membrane (see Non-Patent Document 5, for example). As is clear from these points, this finding was obtained regardless of a plurality of negative teachings in the art relating to cell-membrane penetration, and it is conceded that there was a difficulty in creation.

(2) Subsequently, the inventors of the present invention found that, when a mutant chaperonin complex including the above-mentioned ATP hydrolysis activity-lowered GroEL mutants was synthesized by using “subunits having GroES activity to which a nuclear transport signal peptide (a peptide that enables localization to an intracellular organelle) had been added” as GroES subunits, the chaperonin complex that had been taken up by a cell could target and reach a cell nucleus specifically.

A major technological characteristic relating to the present invention was arrived at by finding that the mutant chaperonin complex including the ATP hydrolysis activity-lowered GroEL subunit mutants and the subunits having GroES activity could be used as carriers in a drug delivery system based on the finding according to the above-mentioned item (1) to realize cell-membrane penetration and local drug delivery into a cell.

Moreover, a further technological characteristic relating to the present invention was arrived at by finding that local drug delivery to an intracellular organelle could be more efficiently realized based on the finding according to the above-mentioned item (2).

The present invention specifically relates to aspects of the invention described below.

[1] A nanocapsule for a drug delivery system including, as a carrier material for encapsulation of a pharmacological component for a nanocapsule for a system of local drug delivery into a cell, a mutant chaperonin complex including an ATP hydrolysis activity-lowered GroEL subunit mutant as a GroEL subunit included in a ring structure and a subunit having GroES activity as a subunit included in an apex portion.

[2] The nanocapsule for a drug delivery system according to aspect 1,

wherein the ATP hydrolysis activity-lowered GroEL subunit mutant is:

(a-1) a GroEL subunit mutant that consists of an amino acid sequence of Sequence ID No. 1,

(a-2) a GroEL subunit mutant that consists of an amino acid sequence obtained through substitution, deletion, and/or addition of one amino acid or two or more amino acids other than alanine at position 398 in the amino acid sequence of Sequence ID No. 1, and exhibits chaperonin activity with extended dissociation half life when a chaperonin complex is formed, or

(a-3) a GroEL subunit mutant that consists of an amino acid sequence including the amino acid sequence of (a-1) or (a-2), and exhibits chaperonin activity with extended dissociation half life when a chaperonin complex is formed.

[3] The nanocapsule for a drug delivery system according to aspect 1,

wherein the ATP hydrolysis activity-lowered GroEL subunit mutant is:

(b-1) a GroEL subunit mutant that consists of an amino acid sequence of Sequence ID No. 2,

(b-2) a GroEL subunit mutant that consists of an amino acid sequence obtained through substitution, deletion, and/or addition of one amino acid or two or more amino acids other than alanines at positions 52 and 398 in the amino acid sequence of Sequence ID No. 2, and exhibits chaperonin activity with extended dissociation half life when a chaperonin complex is formed, or

(b-3) a GroEL subunit mutant that consists of an amino acid sequence including the amino acid sequence of (b-1) or (b-2), and exhibits chaperonin activity with extended dissociation half life when a chaperonin complex is formed.

[4] The nanocapsule for a drug delivery system according to any one of aspects 1 to 3,

wherein the subunit having GroES activity is:

(c-1) a GroES subunit that consists of an amino acid sequence of Sequence ID No. 8,

(c-2) a GroES subunit that consists of an amino acid sequence obtained through substitution, deletion, and/or addition of one amino acid or two or more amino acids in the amino acid sequence of Sequence ID No. 8, that includes a region exhibiting a sequence homology of 70% or more with respect to the amino acid sequence of Sequence ID No. 8, and that exhibits GroES activity when a chaperonin complex is formed,

(c-3) a GroES subunit that consists of an amino acid sequence including the amino acid sequence of (c-1) or (c-2), and exhibits GroES activity when a chaperonin complex is formed,

(d-1) a Gp31 subunit that consists of an amino acid sequence of Sequence ID No. 11,

(d-2) a Gp31 subunit that consists of an amino acid sequence obtained through substitution, deletion, and/or addition of one amino acid or two or more amino acids in the amino acid sequence of Sequence ID No. 11, that includes a region exhibiting a sequence homology of 70% or more with respect to the amino acid sequence of Sequence ID No. 11, and that exhibits GroES activity when a chaperonin complex is formed, or

(d-3) a Gp31 subunit that consists of an amino acid sequence including the amino acid sequence of (d-1) or (d-2), and exhibits GroES activity when a chaperonin complex is formed.

[5] The nanocapsule for a drug delivery system according to any one of aspects 1 to 4, wherein the subunit having GroES activity is a subunit having GroES activity with a peptide for localization to an intracellular organelle added or inserted.

[6] The nanocapsule for a drug delivery system according to aspect 5, which is a nanocapsule for a system of local drug delivery into an intracellular organelle.

[7] The nanocapsule for a drug delivery system according to aspect 5, wherein the peptide for localization to an intracellular organelle is a nuclear transport signal peptide.

[8] The nanocapsule for a drug delivery system according to aspect 7, which is a nanocapsule for a system of local drug delivery into a cell nucleus.

[9] The nanocapsule for a drug delivery system according to any one of aspects 1 to 8, wherein the ATP hydrolysis activity-lowered GroEL subunit mutant is neither subjected to addition or insertion of a peptide including a foreign sequence for selective trans-membrane transport, nor subjected to molecular modification for cell-membrane penetration.

[10] The nanocapsule for a drug delivery system according to aspect 1,

wherein the ATP hydrolysis activity-lowered GroEL subunit mutant is:

(b-1) a GroEL subunit mutant that consists of an amino acid sequence of Sequence ID No. 2,

(b-2) a GroEL subunit mutant that consists of an amino acid sequence obtained through substitution, deletion, and/or addition of one amino acid or two or more amino acids other than alanines at positions 52 and 398 in the amino acid sequence of Sequence ID No. 2, and exhibits chaperonin activity with extended dissociation half life when a chaperonin complex is formed, or

(b-3) a GroEL subunit mutant that consists of an amino acid sequence including the amino acid sequence of (b-1) or (b-2), and exhibits chaperonin activity with extended dissociation half life when a chaperonin complex is formed;

the ATP hydrolysis activity-lowered GroEL subunit mutant is neither subjected to addition or insertion of a peptide including a foreign sequence for selective trans-membrane transport, nor subjected to molecular modification for cell-membrane penetration;

the subunit having GroES activity is:

(c-1) a GroES subunit that consists of an amino acid sequence of Sequence ID No. 8,

(c-2) a GroES subunit that consists of an amino acid sequence obtained through substitution, deletion, and/or addition of one amino acid or two or more amino acids in the amino acid sequence of Sequence ID No. 8, that includes a region exhibiting a sequence homology of 70% or more with respect to the amino acid sequence of Sequence ID No. 8, and that exhibits GroES activity when a chaperonin complex is formed, or

(c-3) a GroES subunit that consists of an amino acid sequence including the amino acid sequence of (c-1) or (c-2), and exhibits GroES activity when a chaperonin complex is formed; and

the subunit having GroES activity is:

a subunit having GroES activity with a peptide for localization to an intracellular organelle added or inserted, and the peptide for localization to an intracellular organelle is a nuclear transport signal peptide.

[11] The nanocapsule for a drug delivery system according to aspect 10, which is a nanocapsule for a system of local drug delivery into a cell nucleus.

[12] The nanocapsule for a drug delivery system according to any one of aspects 1 to 11,

wherein, regarding the GroEL subunits included in the ring structure in the mutant chaperonin complex,

(e-1) all of the GroEL subunits are the ATP hydrolysis activity-lowered GroEL subunit mutants, or

(e-2) half or more of the GroEL subunits are the ATP hydrolysis activity-lowered GroEL subunit mutants, and exhibits chaperonin activity with extended dissociation half life when a chaperonin complex is formed.

[13] The nanocapsule for a drug delivery system according to any one of aspects 1 to 12, including ATPs or alternative compounds of ATP.

[14] The nanocapsule for a drug delivery system according to any one of aspects 1 to 13, containing a pharmacological component inside a ring structure in the mutant chaperonin complex.

[15] The nanocapsule for a drug delivery system according to aspect 14, wherein the pharmacological component is a nucleic acid, a peptide, a protein, modifications thereof or derivatives thereof, or substances containing those compounds.

[16] A method for locally delivering a pharmacological component into a cell, the method using the nanocapsule for a drug delivery system according to any one of aspects 1 to 15.

[17] A method for locally delivering a pharmacological component into a cell, the method including a step of administering the nanocapsule for a drug delivery system according to any one of aspects 1 to 15 to cells under in-vivo or in-vitro conditions.

[18] A medicine including the nanocapsule for a drug delivery system according to aspect 14 or 15.

### Advantageous Effects of the Invention

With the present invention, it is possible to provide a technology that relates to a protein nanocapsule capable of holding a substance to be encapsulated such as a drug and in which the protein nanocapsule can be introduced into a cell using a simple method and the contained substance can reach a target in a cell.

Therefore, with the present invention, it is possible to provide a DDS carrier that is a nanocapsule with a uniform size capable of being used without problems even when passing through a capillary vessel, that is easily modified by the addition of an antibody or the like as it is a protein nanocapsule, and that can penetrate a cell-membrane and carry out local delivery in a cell.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic diagram showing a model of an action mechanism of a chaperonin.

FIG. 2 is a schematic view of a structure near an AhR signal sequence insertion site and a GroES gene in a GroES-NAS expression vector prepared in Example 1. XbaI, NcoI, and NdeI show restriction sites on the vector.

FIG. 3 is a diagram showing a base sequence and an amino acid sequence near the AhR signal sequence insertion site and the GroES gene in the GroES-NAS expression vector prepared in Example 1. In FIG. 3, the amino acid sequence in a white box shows the AhR signal sequence. The amino acid sequence in a gray box shows a sequence corresponding to GroES-WT. Portions indicated by dashed lines respectively show restriction sites of NcoI and NdeI.

FIG. 4 shows a photographic gel image (left diagram) of SDS-PAGE to which the GroES-NAS prepared in Example 1 was subjected, and a signal image (right diagram) of Western blot using an anti-GroES antibody to which the GroES-NAS was subjected. Lane 1: a sample from a small-scale culture without IPTG induction; Lane 2: sample from a small-scale culture with IPTG induction; Lane 3: sample from a large-scale culture with IPTG induction; and Lane 4: GroES-WT (purified protein), which is a wild type.

FIGS. 5A-5B show photographic images of molecular structures of mutant chaperonin complexes prepared in Example 1 taken under a transmission electron microscope (TEM). FIG. 5A is an image showing bullet-shaped complexes. The scale bar in the photograph indicates 50 nm. FIG. 5B is an image showing football-shaped complexes. The scale bar in the photograph indicates 100 nm.

FIG. 6 shows photographic images showing the changes over time taken under a fluorescence microscope when a fluorescence-labeled chaperonin complex containing GFP was added to CHL cells in Example 2. The scale bars in the photographs indicate 20 μm. Sample 2-1 shows a series of images showing the changes in Sample 2-1 over time. Sample 2-2 shows a series of images showing the changes in Sample 2-2 over time.

FIGS. 7A-7B show three-dimensionally stacked cross-sectional images of Sample 2-2 of Example 2 formed by using fluorescence micrographs after a lapse of 48 hours from which CHL cell culture was started. FIG. 7A is an image observed under a fluorescence microscope. FIG. 7B is a stacked cross-sectional image when obliquely viewed from the front side. In FIG. 7, a fault plane in FIG. 7B shows a lateral cross section taken along a white dashed line in FIG. 7A.

FIG. 8 shows photographic gel images of PAGE to which a fluorescence-labeled DNA prepared in Example 3(1) was subjected. Lane F image of pUC19 (template DNA) stained with EtBr; Lane 2: image of amplified non-fluorescence-labeled DNA stained with EtBr; Lane 3; image of amplified fluorescence-labeled DNA stained with EtBr; Lane 1′; fluorescence detection image of pUC19 (template DNA) using an excitation light 460 nm/fluorescence 515 nm filter; Lane 2′; fluorescence detection image of amplified non-fluorescence-labeled DNA using an excitation light 460 nm/fluorescence 515 nm filter; and Lane 3′; fluorescence detection image of amplified fluorescence-labeled DNA using an excitation light 460 nm/fluorescence 515 nm filter.

FIG. 9 shows photographic images showing the results of fluorescence detection as per a stationary time-lapse analysis when mutant chaperonin complexes (Sample 3-1) including AhR-added GroESs that contained gold nanoparticles adsorbing fluorescence-labeled DNA were added to cultured CHL cells and cell culture was performed in Example 3(4). In FIG. 9, the images on the top are composite images of a fluorescent image at excitation light 466 nm/fluorescence 525 nm and a DIC transmission image, and the images on the bottom are DIC transmission images. Numerals shown above the photographic images indicate the time elapsed from when administration of samples was started. Circles shown by dashed lines indicate cell nuclei in cells in which fluorescent signals were observed. The photographic images were taken at 80-fold magnification, and one side of each photographic image corresponds to 255 μm.

FIG. 10 shows enlarged negative photographic images of the composite images of a fluorescent image at excitation light 466 nm/fluorescence 525 nm and a DIC transmission image in FIG. 9 showing the vicinities of the cells in which fluorescent signals were detected.

FIG. 11 shows photographic images showing the results of fluorescence detection as per a stationary time-lapse analysis when mutant chaperonin complexes (Sample 3-2) including wild-type GroESs that contained gold nanoparticles adsorbing fluorescence-labeled DNA were added to cultured CHL cells and cell culture was performed in Example 3(4). Description of the layout of the diagram and the like is the same as that of FIG. 9.

FIG. 12 shows enlarged negative photographic images of the composite images of a fluorescent image at excitation light 466 nm/fluorescence 525 nm and a DIC transmission image in FIG. 11 showing the vicinities of the cells in which fluorescent signals were detected.

FIG. 13 shows photographic images showing the results of fluorescence detection as per a stationary time-lapse analysis when only gold nanoparticles adsorbing fluorescence-labeled DNA (Sample 3-3) were added to cultured CHL cells in Example 3(4). Description of the layout of the diagram and the like is the same as that of FIG. 9.

FIG. 14 shows photographic images showing the results of fluorescence detection as per a stationary time-lapse analysis when cultured CHL cells were cultured without the administration of samples in Example 3(4). Description of the layout of the diagram and the like is the same as that of FIG. 9.

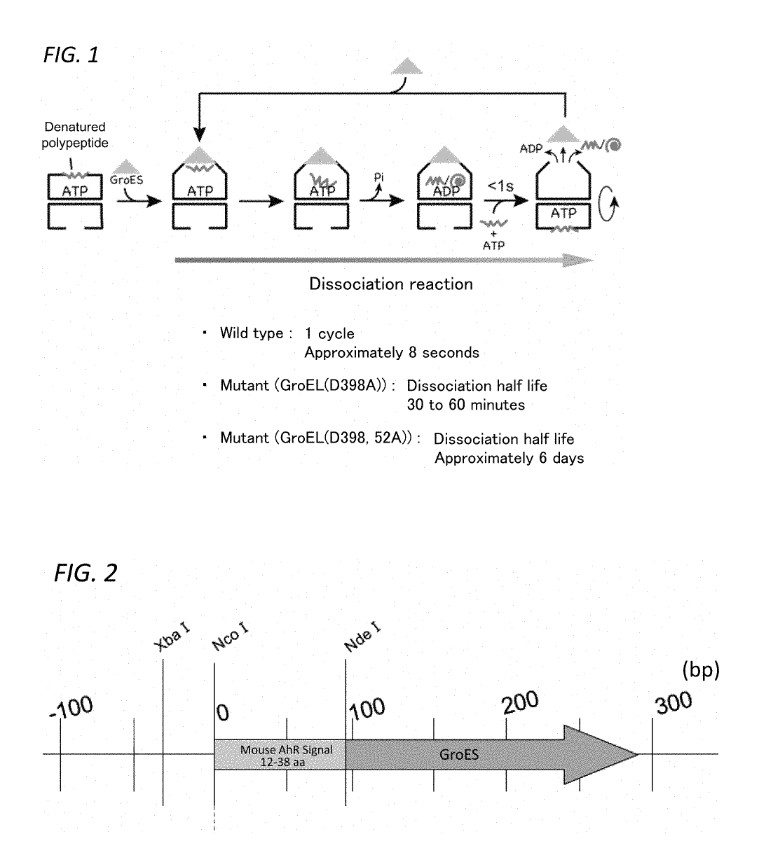

### DESCRIPTION OF EMBODIMENTS

The present application claims priority based on Japanese Patent Application No. 2015-100586, which was filed in Japan on May 16, 2015, by the applicant of the present invention and is hereby incorporated by reference in its entirety.

Hereinafter, embodiments of the present invention will be described in detail.

The present invention relates to a nanocapsule for a drug delivery system that utilizes a mutant chaperonin complex and can carry out local delivery into a cell. The terms “intracellular organelle” and “cellular organelle” as used herein are used as a term that refers to “organelle” as described in common general technical knowledge in the art relating to the invention of the present application on the priority date according to the present application.

1. Mutant Chaperonin Complex

The present invention relates to a technology utilizing, as a carrier material for encapsulation of a pharmacological component for a nanocapsule for a system of local drug delivery into a cell, a mutant chaperonin complex including an ATP hydrolysis activity-lowered GroEL subunit mutant as a GroEL subunit included in a ring structure and a subunit having GroES activity as a subunit included in an apex portion.

The “chaperonin complex” as used herein refers to a nanocapsule-shaped protein having a ring complex structure including a GroEL subunit and a subunit having GroES activity as main constituents.

The GroEL subunits form a heptameric ring structure, and the ring structures are connected back to back to form a tetradecameric double-ring structure. The subunit having GroES activity is connected thereto as an apex capping structure, and thus a three-dimensional structure is obtained that has a closed cavity with a diameter of about 4 to 8 nm (about 5 nm in a case of wild-type E. coli) and that is stable and uniform in an aqueous solution.

A single GroEL subunit consists of an equatorial domain including an ATP binding site, an apical domain including binding sites for a substrate protein and the subunits having GroES activity, and an intermediate domain that connects the equatorial domain and the apical domain. When seven ATPs (including alternative compounds of ATP) bind to the respective chaperonin GroEL subunits forming the ring, the structure of the chaperonin GroEL is changed, thus making it possible for the subunit having GroES activity, which is a cofactor, to bind to the GroEL.

Subsequently, the subunit having GroES activity binds to the GroEL, and the substrate protein (substance to be encapsulated) thus falls into the cavity of the ring, resulting in the formation of a chaperonin complex. In the chaperonin complex, folding of the fallen substrate protein progresses in the cavity of the ring.

When the ATPs (including alternative compounds of ATP) in the ring are hydrolyzed, the subunit having GroES activity dissociates, and the folded substrate protein in the ring dissociates at the same time.

The normal structure of the chaperonin complex according to the present invention is a “bullet-shaped complex” in which a single molecule consisting of the subunits (heptamer) having GroES activity binds to the GroEL tetradecamer, which has a double-ring structure. However, the chaperonin complex according to the present invention also encompasses a “football-shaped complex” in which two molecules each consisting of the subunits (heptamer) having GroES activity bind to the GroEL tetradecamer, which has a double-ring structure.

Moreover, the chaperonin complex according to the present invention also encompasses a complex having a single-ring structure that is a split football-shaped complex. Furthermore, the chaperonin complex according to the present invention also encompasses a complex including, as a subunit, an SR mutant (one type of GroEL mutants) that has a mutation at an interface between the rings and thus inhibits the formation of the double-ring.

The “chaperonin activity” as used in the present application refers to activity that assists in the folding of substrate proteins in an ATP (including alternative compounds of ATP) dependent manner such that the substrate proteins are folded correctly. In particular, regarding the GroEL derived from E. coli, a mechanism for assisting folding of proteins in an ATP and GroES dependent manner has been revealed.

The following are examples of “substitution of an amino acid” as used in the present application. In general, it is preferable to substitute an amino acid with an amino acid having similar characteristics in order to maintain the function of the protein.

Such substitution of amino acids is called conservative substitution. For example, Ala, Val, Leu, Ile, Pro, Met, Phe, and Trp are classified into nonpolar amino acids, and thus have similar characteristics. Examples of non-charged amino acids include Gly, Ser, Thr, Cys, Tyr, Asn, and Gln. Examples of acidic amino acids include Asp and Glu. Examples of basic amino acids include Lys, Arg, and His. Substitution of amino acids in the same group is preferably allowable.

ATP Hydrolysis Activity-Lowered GroEL Subunit Mutant

The mutant chaperonin complex according to the present invention includes an “ATP hydrolysis activity-lowered GroEL subunit mutant” as a GroEL subunit.

In the present invention, the GroEL subunit mutants and the subunits having GroES activity form a stable chaperonin complex (nanocapsule-shaped structure) with a significantly extended dissociation half life, thus realizing penetration of the chaperonin complex into a cell. Since the chaperonin complex is a hydrophilic macromolecular protein, the realization of the penetration of the chaperonin complex into a cell is a surprising finding.

Moreover, in the present invention, the function of releasing an encapsulated substance (e.g., drug) locally in a cell is realized by extending the dissociation half life of the GroEL subunit mutant. Here, “dissociation” of the chaperonin complex as used herein refers to a reaction in which the subunit having GroES activity included in the complex dissociates from the ring structure composed of the GroEL subunits. An encapsulated substance contained in the ring structure of the chaperonin complex is released to the outside of the complex during the dissociation reaction.

Here, the “hydrolysis activity-lowered GroEL subunit mutant” refers to a mutant protein having lower activity for the hydrolysis of ATPs (including alternative compounds of ATP) than a wild-type GroEL. The lowered ATP hydrolysis activity extends the time for dissociation of the subunit having GroES activity from the chaperonin complex, and as a result, the conformation of the complex is maintained for a long period.

Since ATPs hydrolyze in the wild-type GroEL in a very short period of time of about eight seconds, the subunit having GroES activity and the encapsulated substance dissociate immediately. Therefore, it is not preferable to employ a chaperonin complex formed only by the wild-type GroELs as a carrier material for a drug delivery system as it is.

In the mutant chaperonin complex according to the present invention, it is desirable that all of the GroEL subunits forming the heptameric ring structure are preferably ATP hydrolysis activity-lowered subunit mutants. It is also preferable that half or more, preferably 5/7 or more, and more preferably 6/7 or more, of the GroEL subunits forming the ring structure are the ATP hydrolysis activity-lowered GroEL subunit mutants because the conformation of the chaperonin complex can be maintained for a long period of time.

In this regard, in a case where a normal protein expression system of E. coli is used in a process for manufacturing the mutant chaperonin complex according to the present invention, a function for maintaining the conformation of the obtained chaperonin complex for a long period of time is sufficiently exhibited even when wild-type GroEL subunits of E. coli itself are mixed in a small amount (Japanese Patent No. 5540367, Koike-Takeshita et al., J. Biol. Chem. 2014).

Although it is preferable that the mutant chaperonin complex according to the present invention is a tetradecameric complex of the GroEL subunits having a double-ring structure, a heptameric complex of the GroEL subunits having a single-ring structure is also possible. Preferably, a tetradecameric football-shaped complex of the GroEL subunits is favorable to efficiently encapsulate the substance to be encapsulated.

It is sufficient that the ATP hydrolysis activity-lowered subunit according to the present invention is a subunit having lower ATP hydrolysis activity than a wild-type GroEL, and a preferred example thereof is a GroEL (D398A) mutant subunit.

Specifically, the GroEL (D398A) mutant subunit refers to a protein consisting of an amino acid sequence of Sequence ID No. 1 in Sequence Listing. Moreover, this mutant subunit also encompasses a subunit that includes the amino acid sequence of Sequence ID No. 1 and that exhibits chaperonin activity with extended dissociation half life when a chaperonin complex is formed.

The GroEL (D398A) subunit is a GroEL mutant in which aspartic acid (D) at position 398 of the amino acid sequence of the wild-type GroEL is substituted with alanine (A). A cycle time of the reaction including hydrolysis of ATPs is about eight seconds in the wild-type GroEL, but the dissociation half life of the chaperonin complex including this mutant is 30 to 60 minutes (Rye et al., Cell, Vol. 97, 1999).

Similarly, a GroEL subunit mutant that consists of an amino acid sequence obtained through substitution, deletion, and/or addition of one amino acid or two or more amino acids other than alanine at position 398 in the amino acid sequence of Sequence ID No. 1, and that has chaperonin activity, and preferably has chaperonin activity and forms a chaperonin complex having a dissociation half life of 20 minutes or more, preferably 30 minutes or more, more preferably 30 to 120 minutes, and even more preferably 30 to 60 minutes can also be used as the ATP hydrolysis activity-lowered GroEL subunit mutant according to the present invention. Proteins that are GroEL-like subunits derived from bacteria other than E. coli or mutants thereof and satisfy these conditions are also encompassed.

Here, regarding the extent of mutation of the amino acids other than alanine at position 398 in the amino acid sequence of Sequence ID No. 1, it is preferable that the sequence homology is 70% or more, preferably 80% or more, more preferably 90% or more, and even more preferably 95% or more, with respect to the amino acid sequence of Sequence ID No. 1.

It is preferable that the number of mutated sites at which an amino acid is substituted, deleted, or added at positions other than the alanine at position 398 is preferably 100 or less, more preferably 50 or less, even more preferably 25 or less, and even more preferably 10 or less.

Moreover, the subunit mutant also encompasses a subunit that includes a mutated amino acid sequence of Sequence ID No. 1 and that exhibits chaperonin activity with extended dissociation half life when a chaperonin complex is formed.

It is favorable to use a GroEL (D52, 398A) mutant subunit as a more preferred example of the ATP hydrolysis activity-lowered subunit according to the present invention.

Specifically, the GroEL (D52, 398A) mutant subunit refers to a protein consisting of an amino acid sequence of Sequence ID No. 2. Moreover, this mutant subunit also encompasses a subunit that has the amino acid sequence of Sequence ID No. 2 and that exhibits chaperonin activity with extended dissociation half life when a chaperonin complex is formed.

The GroEL (D52, 398A) subunit is a GroEL mutant in which aspartic acid (D) at position 52 is substituted with alanine (A) in the GroEL (D398A) subunit and that has the characteristic of having significantly lowered ATP hydrolysis activity.

The dissociation half life of a chaperonin complex including the GroEL (D52, D398A) subunits is a significantly long period of time of about six days. This is a dramatically high value, which is about 150 to 300 times higher than that of the GroEL (D398A) subunit (Japanese Patent No. 5540367, Koike-Takeshita et al., J. Biol. Chem. 2014).

Here, the inventors of the present invention found for the first time that mutational substitution of alanine at position 52 in a GroEL synergistically reduces the ATP hydrolysis activity.

Similarly, a GroEL subunit mutant that consists of an amino acid sequence obtained through substitution, deletion, and/or addition of one amino acid or two or more amino acids other than alanines at positions 52 and 398 in the amino acid sequence of Sequence ID No. 2, and that has chaperonin activity, and preferably has chaperonin activity and forms a chaperonin complex having a dissociation half life of 2 days or more, preferably 5 days or more, more preferably 5 to 7 days, and even more preferably about 6 days can also be used as the ATP hydrolysis activity-lowered GroEL subunit mutant according to the present invention. Proteins that are GroEL-like subunits derived from bacteria other than E. coli or mutants thereof and satisfy these conditions are also encompassed.

Here, regarding the extent of mutation of the amino acids other than alanines at positions 52 and 398 in the amino acid sequence of Sequence ID No. 2, it is preferable that the sequence homology is 70% or more, preferably 80% or more, more preferably 90% or more, and even more preferably 95% or more, with respect to the amino acid sequence of Sequence ID No. 2.

It is preferable that the number of mutated sites at which an amino acid is substituted, deleted, or added at positions other than alanines at positions 52 and 398 is preferably 100 or less, more preferably 50 or less, even more preferably 25 or less, and even more preferably 10 or less.

Moreover, the subunit mutant also encompasses a subunit that includes a mutated amino acid sequence of Sequence ID No. 2 and that exhibits chaperonin activity with extended dissociation half life when a chaperonin complex is formed.

The ATP hydrolysis activity-lowered GroEL subunit mutant can be prepared using a method of introducing mutation into the wild-type GroEL.

Commonly used methods can be used as a mutation-introducing method without limitation. Examples thereof include a method using PCR, and other genetic engineering methods such as a site-directed mutagenesis kit (manufactured by Stratagene, for example).

A mutation to be introduced may allowably encompass such mutations as those having little effect on the chaperonin activity and ATP hydrolysis activity, neutral mutations, and mutations for adding a separate function to the GroEL subunit mutant of the present invention as long as the above-mentioned functions of the ATP hydrolysis activity-lowered GroEL subunit mutant can be secured.

As the ATP hydrolysis activity-lowered GroEL subunit mutant according to the present invention, the employment of a GroEL subunit mutant that has been subjected to the addition or insertion of a peptide including a foreign sequence for selective trans-membrane transport and/or molecular modification for cell-membrane penetration is not excluded. However, an aspect in which the GroEL subunit is subjected to the addition or the like of a peptide or to molecular modification is not preferred, as it may have influence on the entire three-dimensional structure of a complex to be formed, and as a result, chaperonin activity may be reduced or lost.

Specifically, a chaperonin complex itself that includes the ATP hydrolysis activity-lowered GroEL subunit mutants of the present invention has excellent cell-membrane penetration properties, and therefore, it is preferable that the subunit mutant has been neither subjected to the addition or insertion of a peptide including a foreign sequence for selective trans-membrane transport nor subjected to molecular modification for cell-membrane penetration. This aspect is favorable because it avoids the above-mentioned influence on the entire three-dimensional structure of the complex, and moreover, it avoids difficulty in the preparation of the complex and an increase in cost due to an excess process.

Here, “sequence for selective trans-membrane transport” as used herein refers to an amino acid sequence that exhibits a function of selectively penetrating a cell membrane, and a specific example thereof is an amino acid sequence of a cell-penetrating peptide (CPP). A specific example thereof is an amino acid sequence that exhibits characteristics of being noninvasively taken up by a cell via macropinocytosis, endocytosis, or the like, which are physiological mechanisms of the cell itself, without damage to the cell. It should be noted that the term “foreign” as used herein is used as a term referring to an amino acid sequence other than that of the GroEL subunit.

Moreover, an example of “molecular modification for cell-membrane penetration” is the addition of a boronic acid derivative, but there is no limitation as long as the molecular modification exhibits the characteristics of being noninvasively taken up by a cell without damage to the cell.

Subunit Having GroES Activity

The mutant chaperonin complex according to the present invention includes a subunit having GroES activity as the subunit forming an apex portion.

The main body (a region excluding a peptide for localization to an intracellular organelle) of the subunit having GroES activity is a subunit that has the ability to bind to GroEL and forms the apex portion of the chaperonin complex, and corresponds to a region that serves as a cofactor that causes the complex to exhibit molecular chaperon activity. In general, a heptamer of the subunits having GroES activity forms one molecule, and serves as a cofactor of the GroEL ring structure.

“GroES activity” as used in the present invention refers to activity serving as a cofactor such that a function for forming the apex portion of the chaperonin complex is exhibited due to it having the ability to bind to the GroEL, and thus the complex exhibits molecular chaperon activity.

Any protein can be used as the main body of the subunit that has GroES activity as long as the protein is a GroES-like protein that exhibits the above-mentioned function and activity. Preferred examples thereof include GroES derived from E. coli, GroES homologous proteins derived from bacteria other than E. coli, phage-derived proteins having a GroES-like three-dimensional structure and functions similar to those of GroES, and mutant proteins of these proteins.

Specifically, in the present invention, a wild-type GroES subunit can be used as the main body (a region excluding a peptide for localization to an intracellular organelle) of the subunit having GroES activity. Here, “wild-type GroES subunit” refers to a protein that consists of the amino acid sequence of Sequence ID No. 8 in Sequence Listing. Moreover, this subunit also encompasses a subunit that includes the amino acid sequence of Sequence ID No. 8 and that exhibits the above-mentioned GroES activity when a chaperonin complex is formed.

Similarly, a subunit mutant that consists of an amino acid sequence obtained through substitution, deletion, and/or addition of one amino acid or two or more amino acids in the amino acid sequence of Sequence ID No. 8, and that serves as a constitutional subunit of the chaperonin complex and exhibits the above-mentioned GroES activity when the chaperonin complex is formed can also be used. Proteins that are GroES-like subunits derived from bacteria other than E. coli or mutants thereof and satisfy these conditions are also encompassed.

Here, regarding the extent of mutation of the amino acids in the amino acid sequence of Sequence ID No. 8, it is preferable that the sequence homology is 70% or more, preferably 80% or more, more preferably 90% or more, and even more preferably 95% or more, with respect to the amino acid sequence of Sequence ID No. 8.

It is preferable that the number of mutated sites at which an amino acid is substituted, deleted, or added in Sequence ID No. 8 is preferably 20 or less, more preferably 10 or less, and even more preferably 5 or less.

Moreover, the subunit also encompasses a subunit that includes a mutated amino acid sequence of Sequence ID No. 8 and that exhibits the above-mentioned GroES activity when a chaperonin complex is formed.

Gp31 subunit, which is derived from a wild-type T4 phage, can also be used as the main body (a region excluding a peptide for localization to an intracellular organelle) of the subunit having GroES activity. Here, the Gp31 subunit is a protein that has a three-dimensional structure similar to that of the GroES and is reported as a molecule that forms a chaperonin complex together with the GroEL and exhibits GroES activity. This finding was reported in an academic journal in the art (Hunt et al., Cell 90, 2, (1997) 361-371).

Specifically, the Gp31 subunit refers to a protein that consists of the amino acid sequence of Sequence ID No. 11. Moreover, this subunit also encompasses a subunit that includes the amino acid sequence of Sequence ID No. 11 and that exhibits the above-mentioned GroES activity when a chaperonin complex is formed.

Similarly, a Gp31 subunit mutant that consists of an amino acid sequence obtained through substitution, deletion, and/or addition of one amino acid or two or more amino acids in the amino acid sequence of Sequence ID No. 11, and that serves as a constitutional subunit of the chaperonin complex and exhibits the above-mentioned GroES activity when the chaperonin complex is formed can also be used. Proteins that are Gp31-like subunits derived from phages other than a T4 phage or mutants thereof and satisfy these conditions are also encompassed.

Here, regarding the extent of mutation of the amino acids in the amino acid sequence of Sequence ID No. 11, it is preferable that the sequence homology is 70% or more, preferably 80% or more, more preferably 90% or more, and even more preferably 95% or more, with respect to the amino acid sequence of Sequence ID No. 11.

It is preferable that the number of mutated sites at which an amino acid is substituted, deleted, or added in Sequence ID No. 11 is preferably 20 or less, more preferably 10 or less, and even more preferably 5 or less.

Moreover, the subunit also encompasses a subunit that includes a mutated amino acid sequence of Sequence ID No. 11 and that exhibits the above-mentioned GroES activity when a chaperonin complex is formed.

It is preferable to use “a subunit having GroES activity that has been subjected to the addition or insertion of a peptide for localization to an intracellular organelle” as the GroES subunit included in the mutant chaperonin complex according to the present invention.

The GroES subunit of this aspect is a subunit having GroES activity to which a peptide having the function of localizing the chaperonin complex to an intracellular organelle has been added. With these features, the mutant chaperonin complex according to the present invention realizes local drug delivery into a cell.

In the present invention, “a peptide for localization to an intracellular organelle” specifically refers to a peptide that has the function of transferring a chaperonin complex to a specific intracellular organelle to realize localization of chaperonin complexes to the intracellular organelle. Specific examples of this peptide include (i) a signal peptide, and (ii) a peptide having the ability to bind to a specific protein.

It is not preferable that the above-mentioned GroEL subunit mutant is subjected to the addition or insertion of a peptide because the entire three-dimensional structure of the formed complex is likely to be affected, and the chaperonin activity is likely to be reduced or lost.

(i) It is preferable to use a signal peptide as the peptide for localization to an intracellular organelle according to the present invention. Here, the signal peptide is a peptide that consists of a specific amino acid sequence composed of several amino acid residues to several tens of amino acid residues (about 3 to 60 amino acid residues) and is called a localization signal, a transfer signal, or the like.

In the present invention, any known signal peptide can be used as long as the signal sequence included in the signal peptide exhibits a function for directing localization and transfer of a protein in a cell.

In the present invention, a signal peptide that enables transfer to a specific intracellular organelle can be employed. For example, a peptide including a signal sequence that enables transfer to a nucleus, transfer to a mitochondrial matrix, transfer to an endoplasmic reticulum, transfer to a peroxisome, transfer to a plastid, or the like can be used.

In particular, examples of the nuclear transport signal sequence include a nuclear localization signal sequence (NLS) and a nucleolar localization sequence (NOS), and employing these sequences makes it possible to transfer the chaperonin complex near to or into a cell nucleus and efficiently localize the chaperonin complex near or inside the cell nucleus. Specifically, in an aspect that employs a nuclear transport signal peptide including a signal sequence that enables transfer to a nucleus, the chaperonin complex according to the present invention can be localized near or inside a cell nucleus and transferred near to or into the cell nucleus. There is no limitation on the nuclear transport signal sequence as long as localization near or inside a nucleus or transfer near to or into the nucleus is achieved, and an example thereof is a signal sequence of AhR (aryl hydrocarbon receptor).

(ii) A peptide having the ability to bind specifically to a protein that is localized in a specific intracellular organelle can be used as the peptide for localization to an intracellular organelle according to the present invention.

An example of such a peptide is a peptide that serves as a ligand molecule and binds to a receptor localized in a desired intracellular organelle.

Moreover, a peptide can also be used that has the function of interacting with and binding to a protein localized in a desired intracellular organelle in a molecular specific manner and that participates in the formation of a dimer or a polymer.

It is sufficient that the peptide for localization to an intracellular organelle described in the above (i) and (ii) includes one desired peptide sequence in the main body of the subunit having GroES activity, but two or more peptides can also be employed.

In the chaperonin complex according to the present invention, the peptide for localization to an intracellular organelle can be “added” to a position on the N-terminal side and/or the C-terminal side of the subunit having GroES activity. It is preferable to add the peptide for localization to an intracellular organelle to the N-terminal side of the subunit having GroES activity. Upon adding the peptide for localization to an intracellular organelle, a peptide serving as a linker region can also be provided as long as the three-dimensional structure, functions, and the like are not adversely affected.

Moreover, if the three-dimensional structure, functions, and the like of the subunit having GroES activity are not adversely affected, an aspect is possible in which a peptide sequence is “inserted” at a position other than the N-terminus and C-terminus of this subunit protein.

It is not preferable to add the peptide for localization to an intracellular organelle to the N-terminus or C-terminus of the GroEL (subunit forming the ring structure). The N-terminus and C-terminus of the GroEL project toward the inside of the ring structure, and therefore, even if the above-mentioned peptide is added, any effects cannot be expected in principle.

The peptide for localization to an intracellular organelle can be added to or inserted into the subunit having GroES activity using a common method of synthesizing a fusion protein.

For example, a construct for expressing a fusion protein of GroES and the peptide for localization to an intracellular organelle is constructed using a genetic engineering method, and a fusion protein can be synthesized using the expression vector in E. coli or the like. Moreover, it is possible to prepare the fusion protein as a synthetic protein through polymerization using a chemical method.

When one subunit in the heptamer of the subunits having GroES activity in the mutant chaperonin complex of the present invention is a subunit with a peptide for localization to an intracellular organelle added or inserted, local delivery of an encapsulated substance in a cell is preferably realized.

The mutant chaperonin complex of the present invention may include a plurality of (two or more) the subunits having GroES activity with the peptides for localization to an intracellular organelle added or inserted, but even if only one of the subunits having GroES activity is such a subunit, the chaperonin complex sufficiently exhibits the effect of locally delivering an encapsulated substance in a cell.

Moreover, in a case where a normal protein expression system of E. coli is used in a process for manufacturing the mutant chaperonin complex according to the present invention, the obtained chaperonin complex sufficiently exhibits the effect of locally delivering an encapsulated substance in a cell even when the wild-type GroES subunits of E. coli itself are mixed.

In the present invention, it is preferable that half or more, preferably 5/7 or more, more preferably 6/7 or more, and even more preferably all, of the subunits having GroES activity in the heptamer are subunits with the peptides for localization to an intracellular organelle added or inserted.

ATP Etc.

It is preferable that the chaperonin complex of the present invention includes ATPs or alternative compounds of ATP.

In the chaperonin complex of the present invention, it is particularly preferable to use ATPs, but alternative compounds of ATP can also be used. Here, there is no particular limitation on the alternative compounds of ATP as long as they can bind to an ATP binding site of the GroEL subunit mutant and change the conformation of the chaperonin GroEL mutant.

Examples of alternative compounds of ATP include ADP, a beryllium fluoride adduct of ADP, an aluminum fluoride adduct of ADP, and a gallium fluoride adduct of ADP (J. Biol. Chem., 279, 45737-45743 (2004); J. Mol. Biol., 2003 May 23; 329(1): 121-34). As the alternative compound of ATP, using a compound (e.g., a beryllium fluoride adduct of ADP) that does not hydrolyze at the ATP hydrolysis site of the GroEL makes it possible to keep the chaperonin complex containing an encapsulated substance for a longer period of time.

Other Constitutional Materials Etc.

It is possible to employ aspects of the chaperonin complex according to the present invention that additionally include various constitutional materials for improving the functions of the chaperonin complex as a carrier for a drug delivery system.

For example, a substance to be encapsulated can be contained in the chaperonin mutant more efficiently by further adding metal ions (preferably a magnesium ion) or metal nanoparticles (e.g., FePt, CdS, CdSe, SiO₂, Au) (JP 2013-199457A).

In the chaperonin complex according to the present invention, the surfaces of the ATP hydrolysis activity-lowered GroEL subunit mutants and the subunits having GroES activity can be modified using an antibody or the like in order to ensure the directivity to organs and specific cells.

The ATP hydrolysis activity-lowered GroEL subunit mutants and the subunits having GroES activity according to the present invention also encompass those to which a sugar chain or a fluorescent substance has been added, and those that have undergone molecular modification including substitution of a functional group such as phosphorylation or methylation, as long as the above-mentioned chaperonin activity and functions for delaying ATP hydrolysis are secured.

Preparation of Mutant Chaperonin Complex

The mutant chaperonin complex of the present invention can be formed and prepared (manufactured, produced, or the like) using a GroEL subunit group including the GroEL subunit mutant under normal conditions, for example, in an ATP dependent manner (Nature, 1990 Nov. 22; 348(6299); 339-42).

A specific example is a process for mixing the GroEL subunits including the GroEL subunit mutant and a substance to be encapsulated (e.g., pharmacological component) in a buffer solution, and mixing them so as to come into contact with the subunits having GroES activity and ATPs (including alternative compounds of ATP). Metal ions, metal nanoparticles, or the like can also be mixed and contained therein as desired (JP 2013-199457A).

In the present invention, the chaperonin mutant has lowered ATP hydrolysis activity, so that the state in which the chaperonin complex contains the encapsulated substance (e.g., pharmacological component) can be maintained for a long period of time.

2. Nanocapsule for Drug Delivery System

The mutant chaperonin complex according to the present invention can be utilized as a protein nanocapsule that can hold a substance to be encapsulated such as a drug. Specifically, the mutant chaperonin complex according to the present invention can be utilized as a nanocapsule for a system of drug delivery into a cell. The nanocapsule includes the mutant chaperonin complex according to the present invention as a carrier material for drug delivery.

The mutant chaperonin complex can be used as a carrier material that can contain a pharmacological component inside its ring structure. Specifically, it is possible to realize a nanocapsule for a drug delivery system including a mutant chaperonin complex as a carrier material for encapsulation of a pharmacological component.

The nanocapsule according to the present invention can be utilized as a nanocapsule for a system of local drug delivery into a cell due to the above-mentioned characteristics of the mutant chaperonin complex. In particular, the aspect including the subunits having GroES activity that have been subjected to the addition or insertion of the peptide for localization to an intracellular organelle can be favorably utilized as a nanocapsule for a system of local drug delivery to an intracellular organelle. Furthermore, the aspect including the subunits having GroES activity that have been subjected to the addition or insertion of the nuclear transport signal peptide can be favorably utilized as a nanocapsule for a system of local drug delivery to a cell nucleus.

The mutant chaperonin complex according to the present invention can be formed and manufactured as a complex that contains a pharmacological component in the cavity of the capsule-like structure. Specifically, the mutant chaperonin complex according to the present invention can be formed in the form of a nanocapsule containing a pharmacological component in its ring structure. This form is a protein nanocapsule containing a pharmacological component, and thus can be favorably utilized as a medicine.

Theoretically, any compound of known pharmacological components can be used as the substance to be encapsulated (e.g., pharmacological component) in the present invention as long as it can be contained in the chaperonin mutant. In particular, the chaperonin mutant containing a pharmacological component for cancers, cerebral nerves, or genetic diseases can be favorably utilized as an effective carrier nanocapsule for a drug delivery system.

Specifically, a nucleic acid (e.g., DNA, RNA), a peptide, a protein, a glycoprotein, a polysaccharide, derivatives thereof, modifications thereof or the like can be contained as the pharmacological component, for example. There is no particular limitation on the nucleic acid, and a plasmid, an expression vector, a nucleic acid oligomer, siRNA (small interfering RNA), miRNA (micro RNA), guide RNA for genome editing, a nucleic acid aptamer or the like can be contained, for example. Moreover, there is no particular limitation on the protein and the peptide, and an antibody can also be contained, for example.

As aspects of the pharmacological component, materials that contain the above-mentioned pharmacological component can be similarly encapsulated. Specifically, a mixture or composition containing the pharmacological component, and an adsorbent or conjugate of the pharmacological component and a carrier such as metal nanoparticles can also be employed.

Moreover, a pharmaceutical compound obtained through organic synthesis, a nanocrystal (a nanocrystallized compound), dendrimer nanoparticles or the like can also be contained.

When a low molecule such as a nucleic acid is contained, it is preferable to use a cationic carrier. Examples of the cationic carrier include polyethyleneimine (PEI), chitosan, and poly-L-lysine (PLL), which are positively charged high-molecular polymers. Moreover, metal nanoparticles whose surfaces have been subjected to surface modification using cationic molecules can also be used as the cationic carrier.

The encapsulated substance in the present invention is contained in the chaperonin mutant, and thus, if the substance is a protein, for example, it is desirable to use a protein of 120 kDa or less, preferably 90 kDa or less, and more preferably 60 kDa or less.

In an aspect of the nanocapsule for a drug delivery system according to the present invention that contains a nucleic acid, the nanocapsule is expected to be favorably used in the field of nucleic acid therapy or gene therapy. The nanocapsule for a drug delivery system according to the present invention can be used for delivering a drug to an intracellular organelle localized in cytoplasm, and is particularly expected to be used as a nanocapsule for a system of local drug delivery to a cell nucleus.

The nanocapsule for a drug delivery system according to the present invention can release the encapsulated substance gradually (in its half life of several tens of minutes to several days, for example) as the hydrolysis of ATPs (or alternative compounds of ATP) contained in the mutant chaperonin complex progresses. Such a sustained-release property of the chaperonin complex is a significantly favorable characteristic of a carrier for local drug delivery into a cell.

Moreover, the nanocapsule for a drug delivery system according to the present invention can also release the encapsulated substance at a desired timing. Specifically, the encapsulated substance can be released from the chaperonin complex by reducing the concentration of metal ions (preferably magnesium ions) included in the chaperonin complex using a commonly used method (e.g., a method using a metal chelating compound).

The nanocapsule for a drug delivery system according to the present invention can be used for any cell theoretically as long as the mutant chaperonin complex can penetrate the cell membrane of the cell. Although eukaryotic cells with cellular organelles can be widely used as target cells, the nanocapsule for a drug delivery system according to the present invention can be favorably used for preferably animal cells having no cell wall and the like, and more preferably vertebrate animal cells. In particular, the nanocapsule for a drug delivery system can be favorably used for mammalian cells in the present invention.

The nanocapsule for a drug delivery system according to the present invention can be utilized not only in an in-vivo administration form such as administration through blood vessels, subcutaneous administration, enteric administration, oral administration, and dermal administration but also in a form of in-vitro administration to cultured cells or the like. For example, in the case of administration in in-vitro form, the contained substance can reliably reach a cell nucleus, and thus application to pluripotent stem cells or the like enables utilization in regenerative medicine and the like. Moreover, nuclear transfer ES cells and iPS cells, which are artificially produced pluripotent cells, can also be favorably used as an application target. Furthermore, the nanocapsule for a drug delivery system according to the present invention can be favorably utilized to introduce Yamanaka factors (Oct3/4, Sox2, Klf4, and c-Myc) or the like during the preparation of iPS cells.

The advantage that the contained substance can reliably reach a cell nucleus can also be advantageously utilized in a carrier for gene introduction to be used for research purposes. For example, the nanocapsule for a drug delivery system according to the present invention can also be favorably used as a carrier in genome editing techniques or RNAi.

In the present invention, using the above-mentioned nanocapsule for a drug delivery system according to the present invention makes it possible to realize a method for locally delivering a pharmacological component into a cell (a method for locally delivering a drug into a cell). Specifically, carrying out a process for administering the nanocapsule for a drug delivery system according to the present invention to the above-mentioned cells under in-vivo or in-vitro conditions makes it possible to realize a method for locally delivering a pharmacological component used as the encapsulated substance into a cell.

Furthermore, in the present invention, a method for locally delivering a pharmacological component to an intracellular organelle localized in a cytoplasm can be efficiently realized with some forms of the above-mentioned nanocapsule for a drug delivery system according to the present invention. Moreover, a method for locally delivering a pharmacological component to a cell nucleus can be efficiently realized with some forms of the above-mentioned nanocapsule for a drug delivery system according to the present invention.

### EXAMPLES

Hereinafter, the present invention will be described by use of examples, but the scope of the present invention is not limited by the examples.

### Example 1

### Preparation of Chaperonin Complex Including GroES-NAS

The chaperonin complex including the GroES mutant subunit in which a nuclear transport signal peptide was added to its N-terminus was prepared.

(1) Amplification of Mouse AhR Signal Sequence Oligomer

Synthesized were a sense strand (Sequence ID No. 3) and an antisense strand (Sequence ID No. 4) of an oligomer of 96 bases having a base sequence obtained by respectively adding a restriction enzyme NcoI site and a NdeI site to the 5′-side and 3′-side of a base sequence coding for the amino acid sequence between position 12 and position 38 (the amino acid sequence of Sequence ID No. 7), which is a mouse aryl hydrocarbon receptor (AhR) signal sequence, to be fused to the N-terminus of the GroES.

PCR primers consisting of base sequences of Sequence ID Nos. 5 and 6 were synthesized in order to amplify the mouse AhR signal sequence oligomer to which the restriction enzyme sites were added.

Equal amounts of the above-mentioned sense strand (Sequence ID No. 3) and antisense strand (Sequence ID No. 4) of the mouse AhR signal sequence oligomer were mixed, and the mixture, the amplification primers (Sequence ID Nos. 5 and 6), polymerases, and dNTPs were mixed, heated in advance at 95° C. for 5 minutes, subjected to 25 cycles of a reaction at 95° C. for 30 seconds, 55° C. for 30 seconds and 72° C. for 30 seconds, and then reacted at 72° C. for 7 minutes, using GeneAmp (registered trademark) PCR System 9700 (Applied Bioscience).

The amplified oligomer was inserted into pT7 Blue vector (Takara), and then TA cloning was carried out. E. coli DH5α competent cells were transformed therewith and cultured on an LB/Amp/IPTG/X-gal plate, and then 24 clones were collected through blue-white selection.

Of these clones, 12 clones were cultured in an LB/Amp medium. After cells were collected, plasmids were extracted using QIAprep Spin Miniprep Kit (QIAGEN) and treated using restriction enzymes NcoI and NdeI (Takara) overnight, followed by deactivation of the enzymes through heat processing at 70° C. for 10 minutes.

Electrophoresis was carried out on a 4% agarose gel, and it was confirmed that cut products had lengths of 90 bases and 160 bases, which corresponded to estimated molecular weights.

(2) Construction of GroES-NAS Expression Vector

The mouse AhR signal sequence oligomer that was prepared as described above and underwent restriction enzyme treatment using NdeI and NcoI was subjected to electrophoresis on an agarose gel, and a portion of the gel containing DNA fragments of a desired molecular weight was cut out and subjected to extraction using Wizard SV Gel and PCR Clean-Up System (Promega, Cat. #A9282). In the same manner, GroES N-end His-Tag/pET21(b)+vector that underwent restriction enzyme treatment using NdeI and NcoI was subjected to electrophoresis on an agarose gel, and a portion of the gel containing DNA fragments of a desired molecular weight was cut out and subjected to extraction. The term “pET21(b)+” herein was used as a name of the same vector as “pET-21b(+)”.

The obtained mouse AhR signal sequence oligomer was inserted into the GroES/pET21(b)+vector, and BL21(DE3) competent cells were transformed therewith and cultured. After cultured cells were collected, plasmids were extracted. The extracted plasmids were subjected to restriction enzyme treatment using NdeI and NcoI. Agarose electrophoresis was carried out, and it was confirmed that desired fragments were present at a position corresponding to about 100 base pairs.

The extracted plasmids, T7 Universal Primer, T7 P(24) Primer, T7F Bgl II I UpFs1 Primer, T7 Reverse Primer, BigDye (registered trademark) (Terminator v3.1 Cycle Sequencing Kit, ABI), and Sequencing Buffer (ABI) were mixed, heated in advance at 96° C. for 1 minutes, and subjected to 25 cycles of a reaction at 96° C. for 10 seconds, 50° C. for 5 seconds and 60° C. for 4 minutes, so that the plasmids were amplified. The plasmids were purified using Performa Gel Filtration Cartridges (Edge Bio).

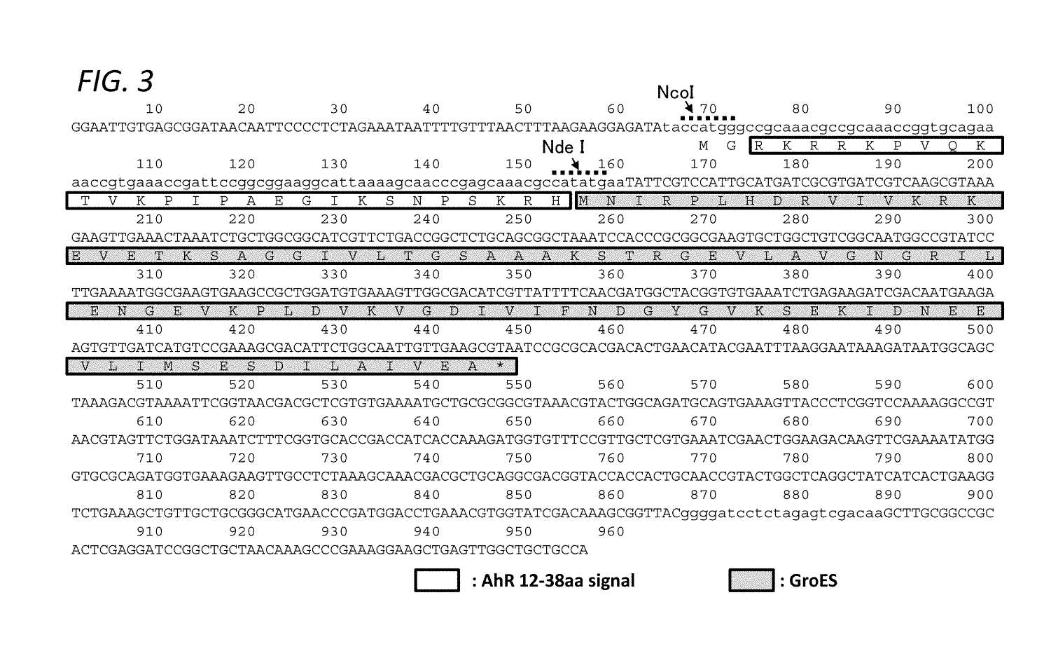

After the reaction solution had dried under vacuum, the dried product was dissolved in Hi-Di formamide, and its base sequence was analyzed using Genetic Analyzer 3130 (ABI). The sequence was read from the 5′-side using T7 Universal Primer and T7P (24) Primer, and it was confirmed that the target mouse AhR signal sequence oligomer was inserted into the GroES/pET21(b)+vector (FIG. 3, Sequence ID No. 10).

FIG. 3 (Sequence ID Nos. 9 and 10) shows the signal sequence and a region coding for GroES in the structure of the prepared construct (also referred to as GroES-NAS/pET21(b)+vector or GroES-NAS expression vector hereinafter), and corresponding amino acids thereof.

(3) Expression and Purification of GroES-NAS Fusion Protein

GroES-NAS was expressed using the above-mentioned expression vector to prepare a fusion protein.

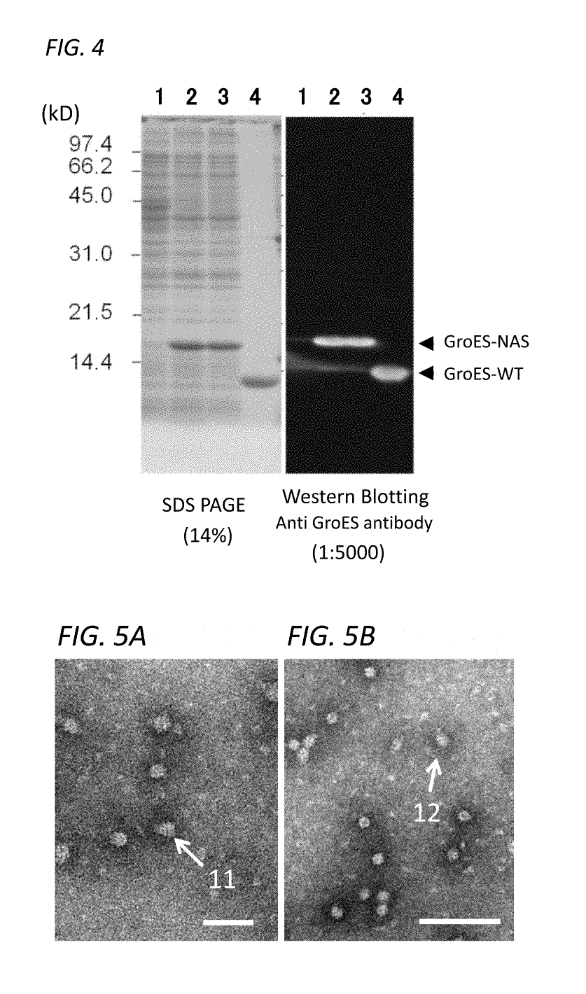

BL21(DE3) that was transformed with the GroES-NAS/pET21(b)+vector was cultured in an LB medium and subjected to IPTG induction at OD=0.8. After collection, cells were sonicated. The supernatant of the centrifuged lysate was used as a sample, and the expression of a fusion protein was confirmed through CBB staining and Western blotting using anti-GroES antibody (FIG. 4).

Next, large-scale culturing of GroES-NAS expression vector/BL21(DE3) was carried out. After collection, cells were sonicated in 20 mM Tris (pH 8.0) containing 1 mM EDTA and centrifuged at 40,000 rpm for 30 minutes. Ammonium sulfate was added to the supernatant to give a 20%-saturated ammonium sulfate solution. The supernatant was ultracentrifuged again, and then applied to Butyl TOYOPEARL M650 (TOSOH) and fractionated with a gradient of 20 to 0% ammonium sulfate.

The obtained elution fraction of the GroES-NAS was placed into a dialysis membrane with a MWCO of 6000 to 8000 and dialyzed in 25 mM citrate buffer solution (pH 4.3) containing 1 mM EDTA. The supernatant of the centrifuged dialyzed sample was applied to SP-TOYOPEARL M650 (TOSOH), and the GroES-NAS was eluted with a gradient of 0 to 1 M NaCl. The elution fraction was concentrated through ultrafiltration using Ultracel (registered trademark)-15(MWCO 10K) (Merck Millipore).

(4) Preparation of GroEL (D52, 398A) Mutant

The GroEL (D52, 398A) mutant, which is an ATP hydrolysis activity-lowered mutant, was prepared according to a method described in Examples in the specification of Japanese Patent No. 5540367. Here, the prepared (D52, 398A) mutant is a protein that consists of the amino acid sequence of Sequence ID No. 2.

(5) Preparation of Chaperonin Complex

The GroES-NAS protein prepared as described above was used to prepare a chaperonin complex at a ratio of 1 μM GroEL/2 μM GroES-NAS/1 mM ATP in a buffer solution of 20 mM HEPES/KOH (pH7.5) (HKM Buffer) containing 100 mM KCl and 5 mM MgCl₂. Here, the GroEL (D52, 398A) mutant prepared as described above was used as GroEL.

Moreover, GroES-WT protein, which is a wild-type GroES, was also used to prepare a chaperonin complex in the same manner as described above.

The chaperonin complex prepared using the GroES-WT (wild type) was taken as sample 1-1, and the chaperonin complex prepared using the GroES-NAS (signal peptide added-type) was taken as sample 1-2. The prepared samples obtained (sample 1-1, sample 1-2) were observed using 6% Native-PAGE and a transmission electron microscope to confirm synthesis of the chaperonin complexes.

(6) Observation of Molecular Structure Under TEM

The molecular structure of the prepared mutant chaperonin complex was observed under a transmission electron microscope (TEM).

The GroEL (D52, 398A) mutant, the GroES-NAS, and ATP were mixed in HKM Buffer to give final concentrations of 0.25 μM, 0.5 μM, and 1 mM, respectively, and the mixture was cooled on ice for 1 hour or longer.

Next, a 400-mesh copper grid with a collodion support film (U1006-400/EM Japan) that underwent hydrophilization treatment using an ion coater was prepared.

Then, 3 μl of a sample solution that had been diluted with ultrapure water to contain 0.1 μM of the chaperonin complex was held on the grid for 30 seconds, and absorbed by a filter paper. Then, 6 μl of ultrapure water was placed thereon and absorbed immediately, and 6 μl of 1% phosphotungstic acid (pH 4.0) was placed thereon for 30 seconds to perform negative staining. The grid after this treatment was dried in a desiccator for 12 hours or longer.

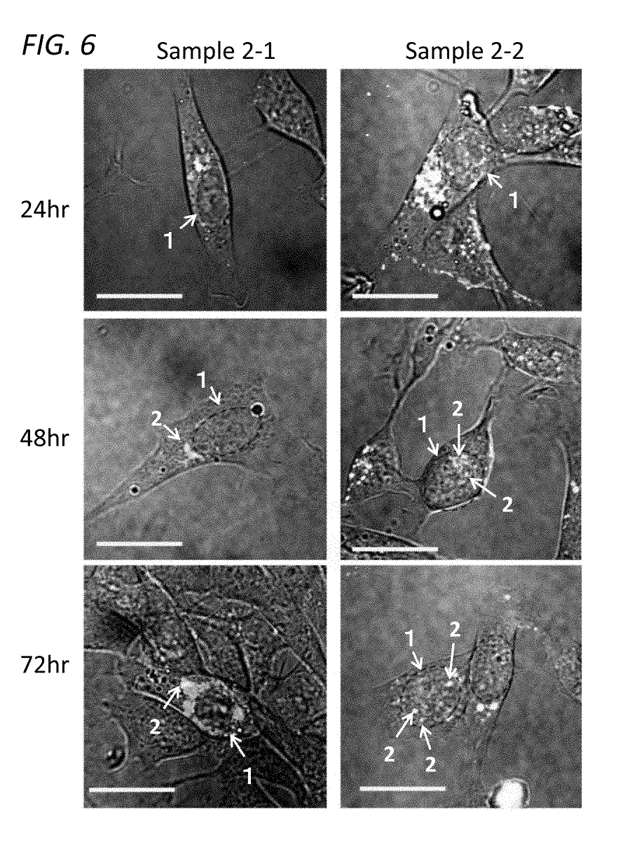

The sample was observed under a transmission electron microscope JEM 1400Plus (JEOL Ltd.) with an acceleration voltage of 80 kV, and a bright field was captured using a CCD camera. FIGS. 5A-5B show the results.

As a result, it was confirmed that the mutant chaperonin complex including GroES-NAS formed a chaperonin complex having a double-ring structure.

Specifically, it was confirmed that a chaperonin complex with a “bullet-shaped” molecular structure including one molecule of GroES-NAS heptamer and a double-ring structure (GroEL tetradecamer) was formed as shown in FIG. 5A. Moreover, it was confirmed that a chaperonin complex with a “football-shaped” molecular structure including two molecules of GroES-NAS heptamer and a double-ring structure (GroEL tetradecamer) was formed as shown in FIG. 5B.

### Example 2

### Introduction of Chaperonin Complex into Mammalian Cells

The cell-membrane penetration properties and the function of local delivery in a cell of the chaperonin complex prepared in Example 1 were examined by carrying out a mammalian cell introduction test using a chaperonin complex containing an encapsulated substance.

(1) Preparation of Chaperonin Complex Containing GFP

A chaperonin complex that included fluorescence-labeled constituent proteins and contained GFP as an encapsulated substance was added to CHL cells, and then observation over time was carried out using a fluorescence confocal microscope.

After the GroES-NAS and the GroES-WT were fluorescently labeled with Cy3 (GE Healthcare), and the GroEL (D52, 398A) was fluorescently labeled with Cy5 (GE Healthcare), the labeled proteins were isolated using a NAP5 gel filtration column (GE Healthcare).

Next, a GFP protein that had been heated at 60° C. for 15 minutes and denatured was caused to be contained in the GroEL, and a chaperonin complex was prepared at a ratio of 2 μM GroEL/4 μM GroES/4 μM GFP/1 mM ATP. Here, the GroEL (D52, 398A) mutant (a protein consisting of the amino acid sequence of Sequence ID No. 2), which is an ATP hydrolysis activity-lowered mutant, was used as the GroEL.

The chaperonin complex prepared using the GroES-WT (wild type) was taken as sample 2-1, and the chaperonin complex prepared using the GroES-NAS (signal peptide added-type) was taken as sample 2-2.

(2) Introduction of Chaperonin Complex into Chinese Hamster Lung (CHL) Cells

After the above-mentioned chaperonin complexes (sample 2-1, sample 2-2) were prepared, a 1/10 volume of 10×HKM Buf was added thereto, and the mixtures were sterilized through filtration using a 0.22-μm membrane filter.

CHL cells were seeded to a φ6-cm dish together with an MEM medium. When the confluence of the cells reached 30%, the chaperonin complex was added thereto to give a final concentration of 0.05 μM in terms of the GroEL. Then, the cells were cultured at 37° C. under 5% CO₂.

Changes in cells over time in both culture states in a culture test to which the chaperonin complex including the GroES-WT (wild type) (sample 2-1) was added and a culture test to which the chaperonin complex including the GroES-NAS (signal peptide-added type) (sample 2-2) was added were observed under a fluorescence confocal microscope FL1000 (OLYMPUS) with triple excitation. FIG. 6 shows photographic images showing the results of the observation over time taken under a fluorescence microscope.

As a result, as shown in the diagrams, fluorescent signals indicating the GFP (green), the GroEL (white) and GroES (red) were observed in the cytoplasm in the culture test to which the chaperonin complex including the GroES-WT (wild type) (sample 2-1) was added.

Here, the proteins such as GFP do not exhibit cell-membrane penetration properties. Moreover, it is reported that a complex structure including the GroEL subunits does not have cell-membrane penetration properties as it is (Biswas et al. 2013). Therefore, the result where the protein included in the chaperonin complex was observed in the cytoplasm is a finding that is contrary to common general technical knowledge assumed from the description in Biswas et al. 2013 (Non-Patent Document 3).

It was deemed that this result was obtained as follows: the formed complex was maintained for a long period of time due to the function of the ATP hydrolysis activity-lowered GroEL (D52, D398A) subunit mutant, and thus a structure capable of penetrating a cell-membrane was maintained for a long period of time. This result is a finding showing, for the first time, that the structure of a chaperonin complex itself has cell-membrane penetrating activity. When a wild-type GroEL subunit complex is formed, the dissociation half life of the complex is a very short period of time of several seconds. Therefore, the complex structure cannot be maintained for a period of time required for local delivery in a cell, and it is thus deemed that delivery of a contained substance in a cell is impossible.

Here, the fluorescent signals were observed only in the cytoplasm in the culture test to which the chaperonin complex including the GroES-WT (wild type) (sample 2-1) was added, and fluorescent signals indicating reaching a nucleus were not obtained even after a lapse of 72 hours. Moreover, GFP, which was used as the encapsulated substance, tended to be released at a slightly earlier timing because the complex itself may be unstable due to the influence of the culture medium and cytoplasm.

Fluorescent signals indicating the GFP (green), the GroEL (white) and GroES (red) were observed in the cytoplasm in the cultured test to which the chaperonin complex including the GroES-NAS (signal peptide-added type) (sample 2-2) was added. Furthermore, a pale yellow signal (signal in which the three fluorescent signals of Cy3, GFP and Cy5 overlapped at the same position) was detected in the nucleus. In particular, a large number of pale yellow signals were observed in the nucleus after a lapse of 48 hours or longer.

It was deemed from the results of detailed observation that the chaperonin complex reached the cytoplasm in 12 to 24 hours, and reached the inside of the nucleus in 36 to 48 hours.

TABLE 1

| | Sample 2-1 | Sample 2-2 |
|---|---|---|
| GroES: Cy3 red | Wild type: GroES-WT | AhR-added: GroES-NAS |
| GroEL: Cy5 | GroEL (D52, 398A) | GroEL (D52, 398A) |
| white | | |
| GFP: Green | GFP | GFP |
| ATP | ATP | ATP |
| Result | Each kind of fluorescent | Many fluorescent |
| | signals was observed in | signals were observed |
| | cytoplasm. | in nucleus. |

(3) Stacked Cross-Sectional Image

In the observation under a fluorescence microscope after a lapse of 48 hours in the above-mentioned introduction test using sample 2-2, one hundred cross-sectional images were captured with a slice interval of 0.1 μm to form a three-dimensional stacked cross-sectional image.

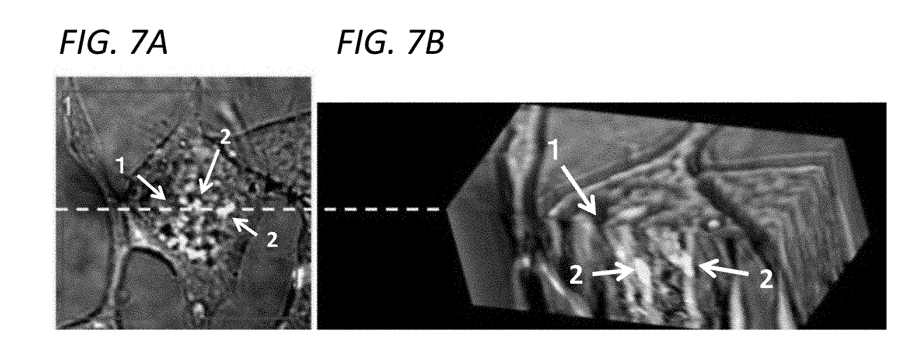

As shown in the three-dimensional image in FIGS. 7A-7B, a large number of pale yellow signals were detected in the nucleus, and it was thus confirmed from the three-dimensional image that the chaperonin complex held the GFP, which was used as an encapsulated substance, even in the nucleus.

(4) Conclusion

It was confirmed from the above-described analysis results that, when the chaperonin complex including a GroEL (D52, 398A) mutant containing the encapsulated substance was used, the chaperonin complex could penetrate a cell membrane and deliver an encapsulated substance to a cytoplasm. It was verified that when the chaperonin complex including a GroEL (D52, 398A) mutant and the nuclear transport signal peptide-added GroESs was used, the encapsulated substance could be delivered into a cell nucleus without decomposing.

### Example 3

### Local Delivery to Cell Nucleus using Mutant Chaperonin Complex Containing Nucleic Acid

It was examined whether or not nucleic acids can be delivered to cell nuclei by carrying out an experimental introduction into mammalian cells using mutant chaperonin complexes containing nucleic acids.

(1) Manufacturing of Fluorescence-Labeled DNA

In a sterilized microtube, 50 μL of a reaction solution having a composition including 0.13 μg/μL pUC19 vector as a template gene (1 μL), 100 μM M13M4 primer (0.5 μL), AmpliTaq Gold 360 Master Mix (25 μL), ChromaTide (registered trademark) Alexa Fluor (registered trademark) 488-5-dUTP (Molecular Probes, Cat. #C-11397) (3.3 μL), and sterilized water (20.2 μL) was prepared. Here, ChromaTide (registered trademark) Alexa Fluor (registered trademark) 488-5-dUTP (Molecular Probes, Cat. #C-11397) is a fluorescence-labeled dUTP that emits a green fluorescence when irradiated with excitation light. AmpliTaq Gold 360 Master Mix contains dNTPs at concentrations suitable for this reaction system.

The mixture solution prepared as described above was heated in advance at 95° C. for 1 minutes, subjected to 40 cycles of a process at 95° C. for 30 seconds, 52° C. for 30 seconds and 72° C. for 30 minutes, and then held at 72° C. for 7 minutes, using 2720 Thermal Cycler (Applied Biosystems). After the reaction was finished, the obtained reaction solution was stored at 4° C.

Moreover, in order to obtain a comparative sample for electrophoresis, an amplification reaction was carried out in the same manner as mentioned above, except that the fluorescence-labeled dUTP was not added.

A sodium dodecyl sulfate (SDS) solution was mixed into the reaction solution to give a final concentration of 0.2%, and SDS treatment through heating at 98° C. for 5 minutes and 25° C. for 10 minutes was carried out using a 2720 Thermal Cycler (Applied Biosystems).

A microtube-type resin column (Performa DTR Gel Filtration Cartridges, Edge Bio, Cat. #4050167) that had been subjected to absorption of 500 μL of sterilized water was centrifuged at 800×g for 3 minutes, and this resin column was placed into a sterilized microtube. Then, 50 μL of the reaction solution that had undergone the SDS treatment was applied to the resin in the column, and centrifugation was carried out at 800×g for 3 minutes to remove unreacted substances. The purified solution eluted from the column was heated and dried using a tabletop vacuum rotor (MicroVac MV-100, TOMY SEIKO Co., Ltd.), and the dried product was redissolved in 50 μL of sterilized water and stored at −25° C., shielded from light.

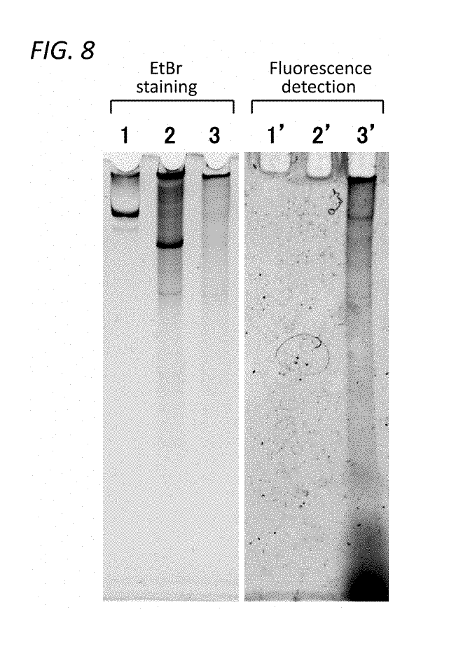

The amplified DNA obtained was subjected to 4.0% PAGE. Electrophoresis was carried out with pUC19 as a template being applied to lane 1, amplified non-fluorescence-labeled DNA being applied to lane 2, and amplified fluorescence-labeled DNA being applied to lane 3. After the electrophoresis was finished, green fluorescence was detected using an excitation light 460 nm/fluorescence 515 nm filter (filter that transmits light having a wavelength of 515 nm or longer). Thereafter, the amplified DNA was confirmed through EtBr staining. FIG. 8 shows the results of captured photographic images of the gel.

As a result, it was confirmed that a fluorescence-labeled DNA fragment was amplified using pUC19 as a template through the above-mentioned cycle reaction, and then purified and collected (FIG. 8: lane 3). Here, although the fluorescence-labeled DNA was single-strand DNA, the stained image obtained using EtBr intercalation was observable due to the association with the template or the formation of a three-dimensional structure.

As shown by the fluorescent signal in lane 3′ in FIG. 8, it was confirmed that the fluorescence-labeled DNA was a DNA fragment that emits a green fluorescence when irradiated with excitation light (FIG. 8: lane 3′). On the other hand, green fluorescence was not detected in the amplified non-fluorescence-labeled DNA (FIG. 8: lane 2′).

(2) Adsorption of Fluorescence-Labeled DNA to Gold Nanoparticles

Into a sterilized microtube, 500 μL of a suspension of gold nanoparticles having an average particle diameter of 2 nm (Spherical Gold Nanoparticles, Nanopartz Inc., Cat. #A-11-2.2) was taken, 10 μL of the fluorescence-labeled DNA solution prepared in the above (1) was added thereto, followed by overnight mixing at 25° C. at 500 rpm using an incubator shaker (Eppendorf ThermoMixer (registered trademark) C).

Sodium acetate and ethanol were added to the obtained suspension to give final concentrations of 0.3 M and 90%, respectively, and the suspension was mixed by inversion and then centrifuged at 14,500 rpm for 5 minutes using a tabletop centrifuge. After the supernatant was removed, the precipitate was resuspended in 50 μL of sterilized water, and thus a suspension of the gold nanoparticles adsorbing the fluorescence-labeled DNA was obtained.

(3) Preparation of Chaperonin Complex Containing Gold Nanoparticles Adsorbing Fluorescence-Labeled DNA

The GroEL (D52, 398A) mutant was added to a buffer solution of HKM Buffer (20 mM HEPES/KOH (pH7.5), 100 mM KCl, 5 mM MgCl₂), and the suspension of the gold nanoparticles adsorbing the fluorescence-labeled DNA prepared in the above (2) was added thereto, followed by mixing by pipetting for 1 minute.

The GroES-NAS (AhR-added type) and ATP were added to this mixture solution, and mutant chaperonin complexes were prepared at a final concentration ratio of 0.5 μM GroEL (D52, 398A)/1.0 μM GroES-NAS/the gold nanoparticles adsorbing the fluorescence-labeled DNA (0.02 mg/mL in terms of gold nanoparticles)/1 mM ATP. These chaperonin complexes were taken as sample 3-1.

The GroES-WT (wild type) and ATP were added to the above-mentioned mixture solution, and mutant chaperonin complexes were prepared at a final concentration ratio of 0.5 μM GroEL (D52, 398A)/1.0 μM GroES-WT/the gold nanoparticles adsorbing the fluorescence-labeled DNA (0.02 mg/mL in terms of gold nanoparticles)/1 mM ATP. These chaperonin complexes were taken as sample 3-2.

The proteins used in this example, that is, the GroEL (D52, 398A) mutant, the GroES-NAS, and the GroES-WT, were prepared in the same manner as in the method described in Example 1.

The obtained solution that contained the chaperonin complexes containing the gold nanoparticles adsorbing the fluorescence-labeled DNA was subjected to ultrafiltration with a centrifugal filter unit (Amicon Ultra-0.5 mL Centrifugal Filters 100KDa, Merck, Cat. #UFC5100BK) using the HKM Buffer to remove excess substances. Ultrafiltration using the centrifugal filter was carried out at 4,000 rpm using a tabletop centrifuge, and the purified and concentrated solution was collected from the filtration membrane through a reverse centrifugation. The obtained solution was stored at 4° C., shielded from light.

(4) Test of Administration to CHL Cells

On a non-coated 35-mm glass bottom dish (IWAKI), 10⁵ CHL cells (fibroblasts derived from a Chinese hamster lung) were seeded, and then cultured in a CO₂ incubator at 37° C. and 5% CO₂ for one day to a state in which the confluence of the cells reached 50%. The mutant chaperonin complexes containing the gold nanoparticles adsorbing the fluorescence-labeled DNA prepared in the above (3) (sample 3-1, sample 3-2) were added thereto to give a final concentration of 0.01 μM in terms of a GroEL concentration.

On the other hand, as a comparative test, the suspension of the gold nanoparticles adsorbing the fluorescence-labeled DNA prepared in the above (2) (sample 3-3) was added to the above-mentioned 50% confluent cells to give a final concentration of 0.0004 mg/mL in terms of the gold nanoparticles, and the culture was carried out in the same manner. The concentration of the gold nanoparticles adsorbing the fluorescence-labeled DNA in this comparative test was adjusted to the same concentration as in the experiments above (sample 3-1, sample 3-2).

After the administration of the sample, the glass bottom dishes were placed into a CO₂ incubator at 37° C. and 5% CO₂, and static culture was carried out for 3 hours. Then, the administered sample was removed by exchanging the culture medium. Thereafter, the glass bottom dishes were placed in an incubator microscope (LCV110-DSU, Olympus) under the conditions of 37° C. and 5% CO₂, and static culture was carried out for 2 hours. Here, the incubator microscope used was provided with a fluorescent cube for GFP (Semrock GFP-4050B), a light source for fluorescence observation (U-HGLGPS, Olympus), a −65° C. cooling CCD camera (Hamamatsu Photonics K.R.), and image analysis software (MetaMorph). The above-mentioned fluorescent cube for GFP is an excitation light 466 nm/fluorescence 525 nm fluorescent filter set including an excitation filter (FF01-466/40-25), a dichroic mirror (FF495-Di03-25x36), a fluorescent filter (FF03-525/50-25), and the like as constituents.

Three stationary observation points per dish were set, DIC transmission images (with an exposure time of 150 milliseconds) and excitation light 466 nm/fluorescence 525 nm fluorescent images (with an exposure time of 200 milliseconds) were captured at 80-fold magnification every 3 hours, and thus images at stationary points after certain periods of time have lapsed from the administration of the sample were obtained. The migration state of the cells continued during static culture, and therefore, cells that were present at the stationary points when observed were captured.

On the other hand, as a comparative test, the static culture was carried out in the same manner as described above, except that the sample was not administered. The exchange of the culture medium and the static culture in the incubator were carried out at a similar timing, and images at the stationary points were captured. It should be noted that, in the comparative test, the start point of a lapse of time was set to the start of the administration in the other sample administration tests, and the elapsed time was measured.

(5) Image Analysis

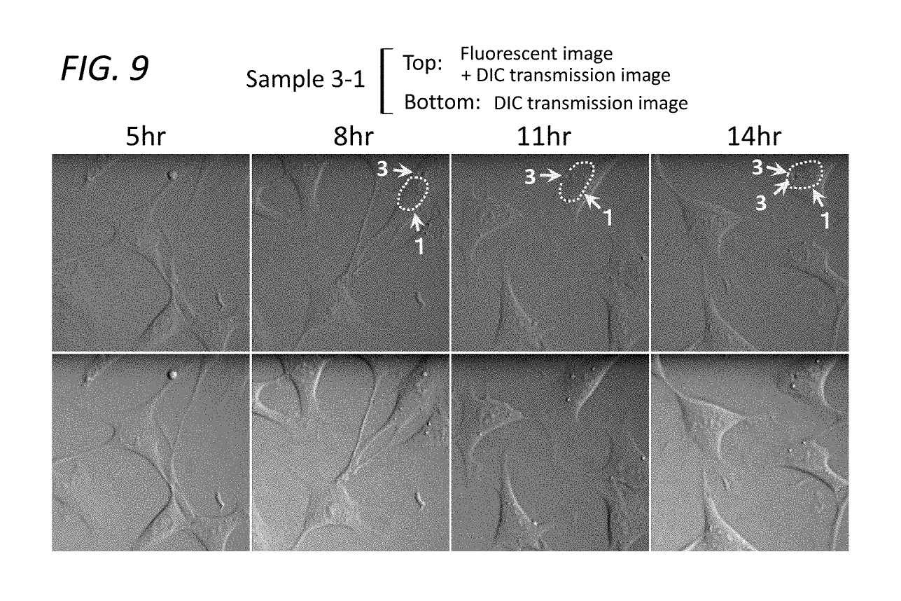

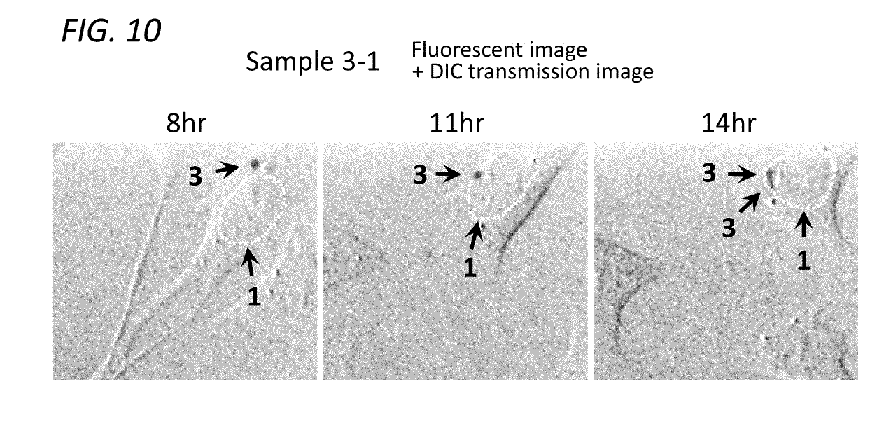

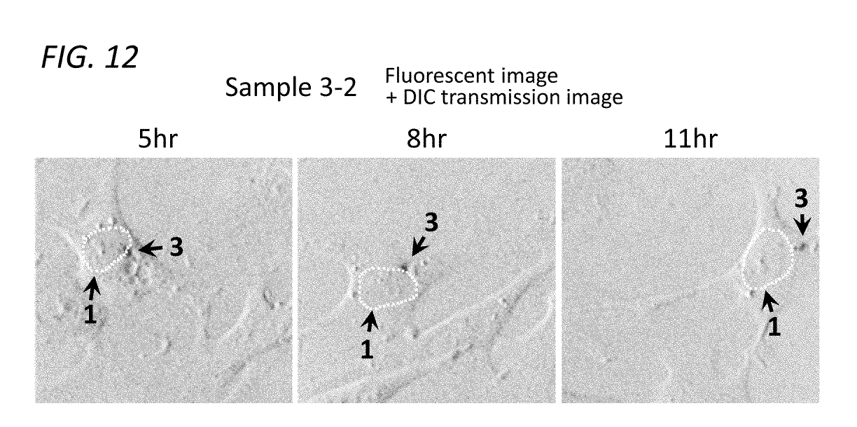

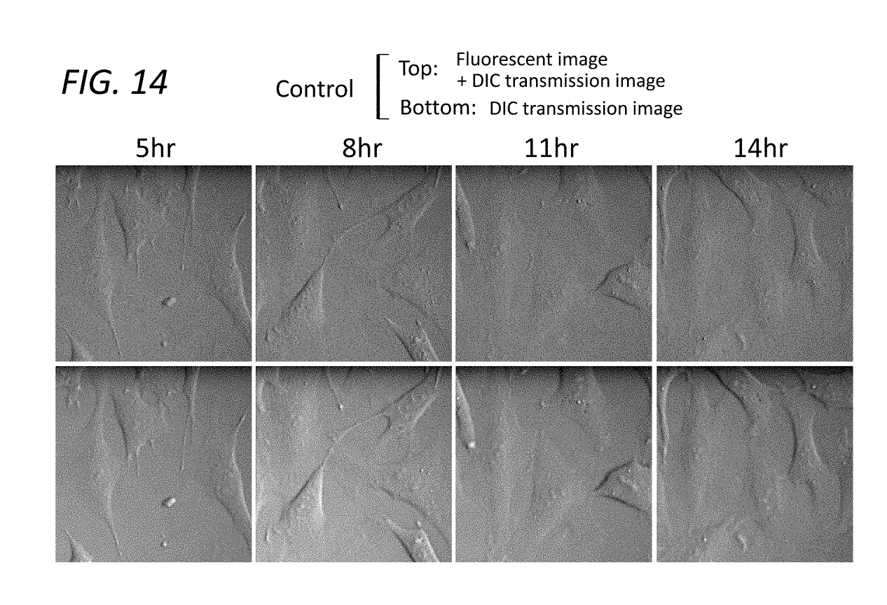

The captured DIC transmission image and fluorescent image were combined to form an overlapping image using image analysis software (MetaMorph). Here, the presence of the fluorescence-labeled DNA prepared in the above (1) can be detected as a fluorescent signal in the image. FIGS. 9 to 14 show the composite images of the DIC transmission image and fluorescent image. FIGS. 10 and 12 show enlarged negative images of the positions at which a fluorescent signal was observed.

As a result, as shown in FIGS. 9 and 10, when the mutant chaperonin complex including the GroES-NASs (AhR-added GroESs) containing the gold nanoparticles adsorbing the fluorescence-labeled DNA (sample 3-1) was added, a fluorescent signal resulting from the fluorescence-labeled DNA was detected in the cytoplasm 8 hours after the addition. Moreover, a plurality of fluorescent signals were detected in the cell nucleus after 11 to 14 hours from the addition.

Here, it was deemed that the contained substance in the mutant chaperonin complex was detected at the positions where the fluorescent signal was detected over time through time-lapse analysis, and it was deemed that the contained substance came closer to the cell nucleus from the cytoplasm as time elapsed, and reached the inside of the nucleus after a lapse of 11 to 14 hours from the addition. Considering that the dissociation half life of the administered mutant chaperonin complex including the GroEL (D52, 398A) is about 6 days, it was inferred that most of the added complexes held the contained substance when they reached the inside of the nucleus.

It was verified from these results that, when the mutant chaperonin complex including the AhR-added GroESs was used, a nucleic acid could be locally delivered into a nucleus without decomposing.

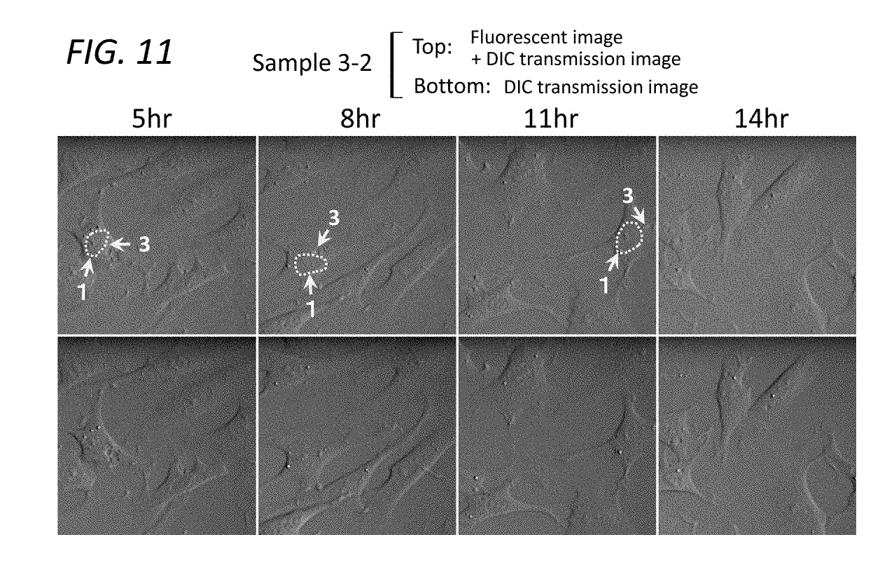

As shown in FIGS. 11 and 12, when the mutant chaperonin complex including the GroES-WTs (wild-type GroESs) containing the gold nanoparticles adsorbing the fluorescence-labeled DNA (sample 3-2) was added, a fluorescent signal resulting from the fluorescence-labeled DNA was detected in the cytoplasm after a lapse of 5 to 11 hours from the addition, but no fluorescent signals were detected in the cell nucleus.

These results did not show that using the mutant chaperonin complex including the wild-type GroESs enabled local delivery into a nucleus. However, the result where the mutant chaperonin complex including the GroEL (D52, 398A) could penetrate a cell membrane and deliver the encapsulated substance into a cytoplasm was a preferred result showing that local delivery into a cell was possible.

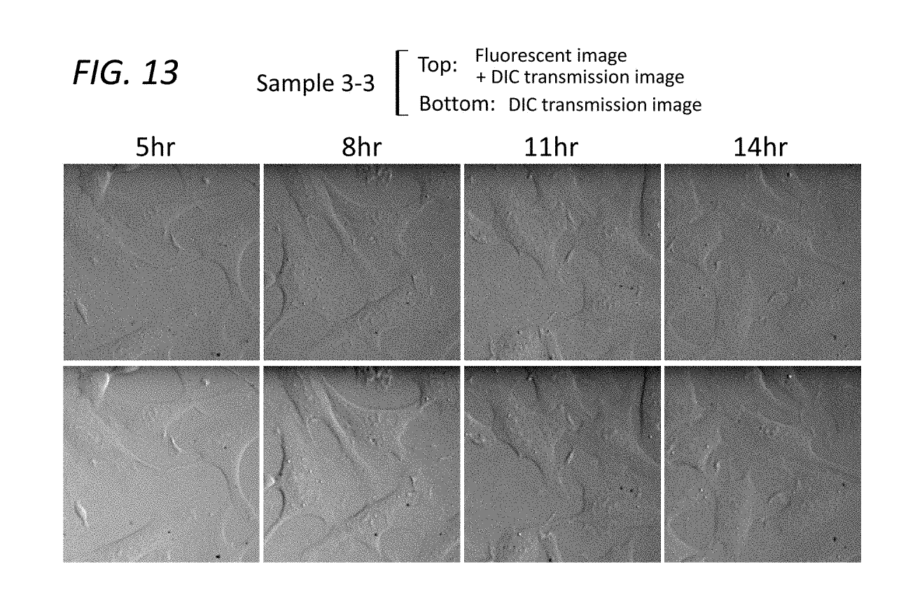

On the other hand, as shown in FIG. 13, in the comparative test in which only the gold nanoparticles adsorbing the fluorescence-labeled DNA (sample 3-3) were added, a fluorescent signal resulting from the fluorescence-labeled DNA was detected in neither the cytoplasm nor the nucleus. It was inferred that the reason for this was that the gold nanoparticles were aggregated and thus were not taken in by a cell.

(6) Conclusion

It was confirmed from the above-described analysis results that, when the mutant chaperonin complex including GroEL (D52, 398A) that contained a nucleic acid molecule was used, the chaperonin complex could penetrate a cell membrane and deliver the nucleic acid molecule to a cytoplasm. It was verified that when the nuclear transport signal peptide-added GroES was further added to the mutant chaperonin complex including GroEL (D52, 398A), the nucleic acid molecule could be delivered into a cell nucleus without decomposing.

TABLE 2

| | | Fluorescence detection | |
|---|---|---|---|
| | | result (time required | |
| | | for detection after sample | |
| | | administration) | |
| | Administration sample | In cytoplasm | In nucleus |
| Experiment | GroEL (D52, 398A)/ | 8 hours | 11 to 14 hours |
| (sample 3-1) | GroES-NAS/gold | | |
| | nanoparticles adsorbing | | |
| | fluorescence-labeled | | |
| | DNA/ATP | | |
| Experiment | GroEL (D52, 398A)/ | 5 to 11 hours | Not detected |
| (sample 3-2) | GroES-WT/gold | | |
| | nanoparticles adsorbing | | |
| | fluorescence-labeled | | |
| | DNA/ATP | | |
| Comparative | Gold nanoparticles | Not detected | Not detected |
| test (sample | adsorbing | | |
| 3-3) | fluorescence-labeled | | |
| | DNA | | |
| Control | Not administered | Not detected | Not detected |

### INDUSTRIAL APPLICABILITY

It is expected that the technology according to the present invention will be an element technology as an organism-derived protein nanocapsule in a system of local drug delivery into a cell. In particular, it is expected to become an important element technology as an intracellular local DDS carrier technology relating to nucleic acid medicine, which is gaining attention in the pharmaceutical industry.

### LIST OF REFERENCE NUMERALS

1: Cell nucleus

2: Pale yellow signal in which GFP, Cy5 and Cy3 overlap

3: Fluorescent signal resulting from fluorescence-labeled DNA

11: Bullet-shaped chaperonin complex

12: Football-shaped chaperonin complex

1. A nanocapsule for a drug delivery system comprising, as a carrier material for encapsulation of a pharmacological component for a nanocapsule for a system of local drug delivery into a cell, a mutant chaperonin complex including an ATP hydrolysis activity-lowered GroEL subunit mutant as a GroEL subunit included in a ring structure and a subunit having GroES activity as a subunit included in an apex portion.

2. The nanocapsule for a drug delivery system according to claim 1,
wherein the ATP hydrolysis activity-lowered GroEL subunit mutant is:
(a-1) a GroEL subunit mutant that consists of an amino acid sequence of Sequence ID No. 1,
(a-2) a GroEL subunit mutant that consists of an amino acid sequence obtained through substitution, deletion, and/or addition of one amino acid or two or more amino acids other than alanine at position 398 in the amino acid sequence of Sequence ID No. 1, and exhibits chaperonin activity with extended dissociation half life when a chaperonin complex is formed, or
(a-3) a GroEL subunit mutant that consists of an amino acid sequence including the amino acid sequence of (a-1) or (a-2), and exhibits chaperonin activity with extended dissociation half life when a chaperonin complex is formed.

3. The nanocapsule for a drug delivery system according to claim 1,
wherein the ATP hydrolysis activity-lowered GroEL subunit mutant is:
(b-1) a GroEL subunit mutant that consists of an amino acid sequence of Sequence ID No. 2,
(b-2) a GroEL subunit mutant that consists of an amino acid sequence obtained through substitution, deletion, and/or addition of one amino acid or two or more amino acids other than alanines at positions 52 and 398 in the amino acid sequence of Sequence ID No. 2, and exhibits chaperonin activity with extended dissociation half life when a chaperonin complex is formed, or
(b-3) a GroEL subunit mutant that consists of an amino acid sequence including the amino acid sequence of (b-1) or (b-2), and exhibits chaperonin activity with extended dissociation half life when a chaperonin complex is formed.

4. The nanocapsule for a drug delivery system according to claim 1,
wherein the subunit having GroES activity is:
(c-1) a GroES subunit that consists of an amino acid sequence of Sequence ID No. 8,
(c-2) a GroES subunit that consists of an amino acid sequence obtained through substitution, deletion, and/or addition of one amino acid or two or more amino acids in the amino acid sequence of Sequence ID No. 8, that includes a region exhibiting a sequence homology of 70% or more with respect to the amino acid sequence of Sequence ID No. 8, and that exhibits GroES activity when a chaperonin complex is formed,
(c-3) a GroES subunit that consists of an amino acid sequence including the amino acid sequence of (c-1) or (c-2), and exhibits GroES activity when a chaperonin complex is formed,
(d-1) a Gp31 subunit that consists of an amino acid sequence of Sequence ID No. 11,
(d-2) a Gp31 subunit that consists of an amino acid sequence obtained through substitution, deletion, and/or addition of one amino acid or two or more amino acids in the amino acid sequence of Sequence ID No. 11, that includes a region exhibiting a sequence homology of 70% or more with respect to the amino acid sequence of Sequence ID No. 11, and that exhibits GroES activity when a chaperonin complex is formed, or
(d-3) a Gp31 subunit that consists of an amino acid sequence including the amino acid sequence of (d-1) or (d-2), and exhibits GroES activity when a chaperonin complex is formed.

5. The nanocapsule for a drug delivery system according to claim 1, wherein the subunit having GroES activity is a subunit having GroES activity with a peptide for localization to an intracellular organelle added or inserted.

6. The nanocapsule for a drug delivery system according to claim 5, which is a nanocapsule for a system of local drug delivery into an intracellular organelle.

7. The nanocapsule for a drug delivery system according to claim 5, wherein the peptide for localization to an intracellular organelle is a nuclear transport signal peptide.

8. The nanocapsule for a drug delivery system according to claim 7, which is a nanocapsule for a system of local drug delivery into a cell nucleus.

9. The nanocapsule for a drug delivery system according to claim 1, wherein the ATP hydrolysis activity-lowered GroEL subunit mutant is neither subjected to addition or insertion of a peptide including a foreign sequence for selective trans-membrane transport, nor subjected to molecular modification for cell-membrane penetration.

10. The nanocapsule for a drug delivery system according to claim 1,
wherein the ATP hydrolysis activity-lowered GroEL subunit mutant is:
(b-1) a GroEL subunit mutant that consists of an amino acid sequence of Sequence ID No. 2,
(b-2) a GroEL subunit mutant that consists of an amino acid sequence obtained through substitution, deletion, and/or addition of one amino acid or two or more amino acids other than alanines at positions 52 and 398 in the amino acid sequence of Sequence ID No. 2, and exhibits chaperonin activity with extended dissociation half life when a chaperonin complex is formed, or
(b-3) a GroEL subunit mutant that consists of an amino acid sequence including the amino acid sequence of (b-1) or (b-2), and exhibits chaperonin activity with extended dissociation half life when a chaperonin complex is formed;
the ATP hydrolysis activity-lowered GroEL subunit mutant is neither subjected to addition or insertion of a peptide including a foreign sequence for selective trans-membrane transport, nor subjected to molecular modification for cell-membrane penetration;
the subunit having GroES activity is:
(c-1) a GroES subunit that consists of an amino acid sequence of Sequence ID No. 8,
(c-2) a GroES subunit that consists of an amino acid sequence obtained through substitution, deletion, and/or addition of one amino acid or two or more amino acids in the amino acid sequence of Sequence ID No. 8, that includes a region exhibiting a sequence homology of 70% or more with respect to the amino acid sequence of Sequence ID No. 8, and that exhibits GroES activity when a chaperonin complex is formed, or
(c-3) a GroES subunit that consists of an amino acid sequence including the amino acid sequence of (c-1) or (c-2), and exhibits GroES activity when a chaperonin complex is formed; and
the subunit having GroES activity is:
a subunit having GroES activity with a peptide for localization to an intracellular organelle added or inserted, and the peptide for localization to an intracellular organelle is a nuclear transport signal peptide.

11. The nanocapsule for a drug delivery system according to claim 10, which is a nanocapsule for a system of local drug delivery into a cell nucleus.

12. The nanocapsule for a drug delivery system according to claim 1,
wherein, regarding the GroEL subunits included in the ring structure in the mutant chaperonin complex,
(e-1) all of the GroEL subunits are the ATP hydrolysis activity-lowered GroEL subunit mutants, or
(e-2) half or more of the GroEL subunits are the ATP hydrolysis activity-lowered GroEL subunit mutants, and exhibits chaperonin activity with extended dissociation half life when a chaperonin complex is formed.

13. The nanocapsule for a drug delivery system according to claim 1, comprising ATPs or alternative compounds of ATP.

14. The nanocapsule for a drug delivery system according to claim 1, containing a pharmacological component inside a ring structure in the mutant chaperonin complex.

15. The nanocapsule for a drug delivery system according to claim 14, wherein the pharmacological component is a nucleic acid, a peptide, a protein, modifications thereof or derivatives thereof, or substances containing those compounds.

16. A method for locally delivering a pharmacological component into a cell, the method using a nanocapsule for a drug delivery system comprising, as a carrier material for encapsulation of a pharmacological component for a nanocapsule for a system of local drug delivery into a cell, a mutant chaperonin complex including an ATP hydrolysis activity-lowered GroEL subunit mutant as a GroEL subunit included in a ring structure and a subunit having GroES activity as a subunit included in an apex portion.

17. A method for locally delivering a pharmacological component into a cell, the method comprising a step of administering a nanocapsule for a drug delivery system comprising, as a carrier material for encapsulation of a pharmacological component for a nanocapsule for a system of local drug delivery into a cell, a mutant chaperonin complex including an ATP hydrolysis activity-lowered GroEL subunit mutant as a GroEL subunit included in a ring structure and a subunit having GroES activity as a subunit included in an apex portion.

18. A medicine comprising a nanocapsule for a drug delivery system comprising, as a carrier material for encapsulation of a pharmacological component for a nanocapsule for a system of local drug delivery into a cell, a mutant chaperonin complex including an ATP hydrolysis activity-lowered GroEL subunit mutant as a GroEL subunit included in a ring structure and a subunit having GroES activity as a subunit included in an apex portion, the nanocapsule containing a pharmacological component inside a ring structure in the mutant chaperonin complex.


SEQUENCE LISTING (11 sequences)

SEQ ID NO: 1 - protein, 548 aa, Artificial Sequence
  GroEL mutant
  MISC_FEATURE: GroEL(D398A) mutant
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEAALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMGGMGGMM

SEQ ID NO: 2 - protein, 548 aa, Artificial Sequence
  GroEL mutant
  MISC_FEATURE: GroEL(D52, 398A) mutant
MAAKDVKFGNDARVKMLRGVNVLADAVKVTLGPKGRNVVLDKSFGAPTITKAGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEAALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMGGMGGMM

SEQ ID NO: 3 - DNA, 96 bp, Artificial Sequence
  oligonucleotide
  misc_feature: AhR signal with REsite forward seq.
taccatgggccgcaaacgccgcaaaccggtgcagaaaaccgtgaaaccgattccggcggaaggcattaaaagcaacccgagcaaacgccatatgaa

SEQ ID NO: 4 - DNA, 96 bp, Artificial Sequence
  oligonucleotide
  misc_feature: AhR signal with REsite reverse seq.
ttcatatggcgtttgctcgggttgcttttaatgccttccgccggaatcggtttcacggttttctgcaccggtttgcggcgtttgcggcccatggta

SEQ ID NO: 5 - DNA, 25 bp, Artificial Sequence
  PCR primer
  misc_feature: AhR forward primer
ttcatatggcgtttgctcgggttgc

SEQ ID NO: 6 - DNA, 25 bp, Artificial Sequence
  PCR primer
  misc_feature: AhR reverse primer
taccatgggccgcaaacgccgcaaa

SEQ ID NO: 7 - protein, 27 aa, Mus musculus
  MISC_FEATURE: AhR signal
RKRRKPVQKTVKPIPAEGIKSNPSKRH

SEQ ID NO: 8 - protein, 97 aa, Escherichia coli [sequence omitted]

SEQ ID NO: 9 - protein, 126 aa, Artificial Sequence
  fused protein
  MISC_FEATURE: GroES-NAS(Mouse AhR signal12-38aa fused N-end of GroES)
MGRKRRKPVQKTVKPIPAEGIKSNPSKRHMNIRPLHDRVIVKRKEVETKSAGGIVLTGSAAAKSTRGEVLAVGNGRILENGEVKPLDVKVGDIVIFNDGYGVKSEKIDNEEVLIMSESDILAIVEA

SEQ ID NO: 10 - DNA, 956 bp, Artificial Sequence
  construct sequence
  misc_feature: GroES-NAS(Mouse AhR signal12-38aa fused N-end of GroES)
ggaattgtgagcggataacaattcccctctagaaataattttgtttaactttaagaaggagatataccatgggccgcaaacgccgcaaaccggtgcagaaaaccgtgaaaccgattccggcggaaggcattaaaagcaacccgagcaaacgccatatgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaatccgcgcacgacactgaacatacgaatttaaggaataaagataatggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttacggggatcctctagagtcgacaagcttgcggccgcactcgaggatccggctgctaacaaagcccgaaaggaagctgagttggctgctgcca

SEQ ID NO: 11 - protein, 111 aa, Enterobacteria phage T4
  MISC_FEATURE: Gp31
MSEVQQLPIRAVGEYVILVSEPAQAGDEEVTESGLIIGKRVQGEVPELCVVHSVGPDVPEGFCEVGDLTSLPVGQIRNVPHPFVALGLKQPKEIKQKFVTCHYKAIPCLYK